(19) United States
(12) Patent Application Publication
(10) Pub. No.: US 2019/0001133 A1
(43) Pub. Date: Jan. 3, 2019

(54) TRANSCRANIAL ELECTRICAL STIMULATION DEVICE HAVING MULTIPURPOSE ELECTRODES

(71) Applicant: Copenhagen Institute of Neurocreativity Devices IVS, Copenhagen (DE)

(72) Inventors: Balder Onarheim, Copenhagen (DK); Morten Friis-Olivarius, Knäred (SE); Ian Jørgensen, Vedbæk (DK); Mads Ring, Hørsholm (DK); Simon Madsen, Copenhagen (DK); Nanna Jordahn Kristoffersen, Copenhagen (DK)

(21) Appl. No.: 16/064,373

(22) PCT Filed: Dec. 22, 2016
(86) PCT No.: PCT/DK2016/050456

§ 371 Date: Jun. 20, 2018

(30) Foreign Application Priority Data
Dec. 22, 2015 (DK) PA 2015 00835

(51) Int. Cl. A61N 1/36; A61N 1/04

(52) U.S. Cl. CPC A61N 1/36025; A61N 1/0456; A61B 5/6843; A61N 1/36034; A61B 2560/0223; A61N 1/36031

(57) ABSTRACT

The present invention relates to an electrotherapy stimulation device having at least three fully programmable multipurpose electrodes in fixed positions and the use of such device for achieving various cognitive effects such as those involved in creative problem solving. The electrodes of the present invention are multipurpose electrodes designed so each electrode can selectively serve multiple functions such as, but not limited to, as anode, cathode or ground of a stimulation circuit.

### FIELD OF THE INVENTION

The present invention relates to an electrical stimulation device having multipurpose electrodes on fixed locations and the use of such device for achieving various cognitive effects such as those involved in creative problem solving.

### BACKGROUND OF THE INVENTION

Transcranial Electrical Stimulation (TEM) is a non-invasive technique where brain regions are targeted using arrays of electrodes on the scalp. It has been shown that TEM can modulate cortical excitability and spontaneous firing activities in the stimulated region by shifting the resting membrane potential. Depending on the polarity and the current of the flow, cortical excitability can be increased (anodal stimulation) or decreased (cathodal stimulation) both during and beyond the period of stimulation.

US 2015/0258327 relates to a cranial electrotherapy stimulation (CES) device having relatively flexible structures that are suitable for various head sizes and stimulation points on the subject's head.

U.S. Pat. No. 9,002,458 relates to a device for TEM comprising two electrodes where the electrical stimulation comprises an alternating current in a specific current and a specific pulse length.

U.S. Pat. No. 8,903,494 describes a two-part wearable device for transdermal electrical stimulation comprising electrodes adhesively attached to the scalp of a user and a power distribution device configured to deliver an electric impulse of a frequency of at least 640 Hz in order to induce a cognitive effect.

U.S. Pat. No. 9,014,811 describes a method of modifying a subject's cognitive state by applying a pulsed, asymmetric, bi-phased current to the surface of the subject's head through two electrodes attached thereto.

U.S. Pat. No. 8,554,324 discloses a neuro electric stimulation device comprising a monitoring and safety device configured to log values of electricity applied to the subject's brain, and to store it in a digital file.

WO2013/113059 discloses a device for stimulating the brain in order to increase the user's ability in creative tasks and insight related tasks, where the device comprises an anode and a cathode to be attached to the skin of the user's head. Thus, WO 2013/113059 describes a two-electrode application of electrical stimulation for left-right hemisphere stimulation, targeting the functionality of “idea generation and insight related tasks”. However, the device does not have three or more electrodes or any programmable multipurpose electrodes.

EP2533850 relates to an apparatus configured to increase the numerical ability at users of the device by stimulating the user's brain with electrical current.

WO2009/137683 describes a device transcranial electrical stimulation of a patient's brain, configured to be self-contained for the user and having an adjustable cap enabled to be fitted to a variety of head sizes.

EP 2524649 describes a two-electrode EEG and TES device, capable of reading brain signals using EEG, and electrical stimulation of the brain using TES. This disclosure only includes the usage of two predefined electrodes and thus does not contain three or more multipurpose electrodes rendering the device not programmable.

US 2014/0018881 describes the electrical wiring of a printed circuit board for two-electrode tDCS, however this application does not disclose three or more electrodes nor is there any disclosure of programmable multipurpose electrodes.

US 2013/0079659 describes a headset primarily for integrating a form of TES, transcranial direct current stimulation (tDCS), and electrical brain signal reading (electroencephalography (EEG)). The application defines a set number of electrodes and utilizes ‘high definition tDCS’ (HD-tDCS) using significantly smaller electrodes for a different type of tDCS application. Furthermore, the disclosure focuses on the usage of ‘pairs of electrodes’ for applying the tDCS, not three or more electrodes in the stimulation, and does not specify the left-right placement for stimulation. The disclosure does not describe multipurpose electrodes, and it does not include a wireless control unit.

US 2015/0005840 describes a two-electrode application of TES, for a given type of cognitive enhancement (relaxation or focus) through electrodes adhesively attached to the scalp of a user. Changing the function of the device requires changing electrodes and electrode placement manually, and the disclosure does not specify e.g. left-right hemisphere placements. Thus, the application does not describe three or more multipurpose electrodes in one montage, rendering the device not programmable.

Conventional tDCS apply singular currents in one direction (anode to cathode) to stimulate a certain effect, such as relaxation (www.Thync.com) or lucid dreaming (www.foc.us). In these existing systems, all electrodes are connected to the electrical system and they will therefore have either to be disconnected from the user's head, or physically removed from the electrical circuit, to be deactivated.

So the electrical brain stimulation electrodes currently available are wired to offer a certain polarity per session, limiting the functionality of the product to only offer a predetermined current direction and location.

### SUMMARY OF THE INVENTION

In the existing devices, changing the mode of the stimulation will require a physical change in wiring and/or physical replacement of the electrodes on the user's head. With a number of multipurpose electrodes attached to the skin, the type of stimulation can be changed instantly and advanced predefined stimulation protocols can be programmed and initiated without changing the wiring or the placement. The fixed placement of at least three electrodes is a benefit of the present invention, as the device can induce different stimulation programs without replacing electrodes. This differentiates the system from any two-electrode devices available, where only one stimulation per montage is possible.

The present invention relates to a system for applying transcranial electrical stimulation (TES) using a weak electrical current applied to a human brain with a device that ensures a rapid switch in the flow, and multiple simultaneous flows and opposite flows through at least three multipurpose electrodes: anode, cathode, deactivation and/or ground.

One or more of the multipurpose electrodes of the present invention can also be completely disconnected from the electrical circuit in the product, so they have no functionality even though they are still in connection with the user's head.

The present invention further comprises complex flows involving a continuous switch of flow between multipurpose electrodes and combinations of various types of TES such as, but not limited to, tDCS, tACS, tRNS, and tPCS.

The wiring of the multipurpose electrodes offers an immediate switch between the polarity of the electrodes, or disconnection of an electrode from the electrical circuit. Thus, the system for applying a weak electrical current according to the present invention allows a range of currents to be programmed, and single electrodes can be disconnected completely from the circuit.

The present invention is the first system to apply transcranial electrical stimulation by use of a multipurpose electrode comprising a ground function and the option to selectively activate and deactivate multiple electrodes in programmed sequences. Adding this multipurpose effect is not simple, as it requires radically different wiring and electronics for all three or more multipurpose electrodes, compared to existing known inventions. But the benefit of three or more programmable multipurpose electrodes is decisive, as it allows for programming a wide range of different TES stimulation protocols using the same device and without replacing the location of electrodes in the middle of a stimulation protocol.

Another embodiment of the present invention is, the basic fixed placement and a flexible placement of the multipurpose electrodes for achieving the desired cognitive effects.

The placement of the multipurpose electrodes, combined with the ability to instantly shift the polarity and/or the connectivity of the electrodes, gives the possibility to stimulate several different areas in a given sequence to induce the desired physical effect.

In the present invention, the flow of the electrical current can be changed instantly to move the stimulation from one brain area to another. This enables the skilled addressee to perform rapid shifts, and the ability to stimulate the same brain area in different ways, using different (or no) polarity during these rapid shifts.

Thus, in one embodiment, the system of the present invention encompasses at least three multipurpose electrodes, wherein each electrode is wired to interchangeably provide anodal stimulation, cathodal stimulation, deactivation, and/or ground, and a distribution unit configured to provide the anodal stimulation, cathodal stimulation, deactivation and/or ground, wherein the electrodes are positioned in a fixed pattern, so that at least two of the electrodes can be disposed on the right hemisphere, and at least one can be disposed on the left hemisphere.

In one aspect, the present invention relates to a system for applying a weak electrical current to a human brain and/or nervous tissue, wherein the system comprises
a) at least three multipurpose electrodes applied in a fixed position on the head of a human subject, wherein each electrode is wired to interchangeably function as anodal stimulation, cathodal stimulation, deactivation, and/or ground,
b) a distribution unit configured to provide the anodal stimulation, cathodal stimulation, deactivation, and/or ground, and

wherein at least two of the electrodes are disposed on the right hemisphere and at least one is disposed on the left hemisphere.

In another aspect, the present invention relates to a system for transcranial electrical stimulation (TES) by applying a weak electrical current to a human brain and/or nervous tissue, wherein the system comprises
a) at least three multipurpose electrodes, wherein each electrode is wired to interchangeably function as anodal stimulation, cathodal stimulation, and/or ground, or be disconnected from the electrical circuit
b) a distribution unit configured to provide the anodal stimulation, cathodal stimulation, and/or ground, and disconnection of electrodes, and

wherein at least two of the electrodes are disposed to be placed on the right hemisphere and at least one is disposed to be placed on the left hemisphere.

Another aspect relates to the use of the system for inducing a cognitive effect in a human subject by stimulating the brain using a weak electrical current applied to the surface of the head of the subject by multipurpose electrodes configured to distribute electric energy to said electrodes according to a determined sequence, pattern or signal.

### DETAILED DESCRIPTION OF THE INVENTION

The present invention relates to a system for applying a weak electrical current to a human brain and/or nervous tissue, wherein the system comprises
a) at least three electrodes applied on the head of a human subject, wherein each electrode is wired to interchangeably function as anodal stimulation, cathodal stimulation, deactivation, and/or ground, and
b) a distribution unit configured to provide the anodal stimulation, cathodal stimulation, deactivation, and/or ground, and

wherein at least two of the electrodes are disposed on the right hemisphere and at least one is disposed on the left hemisphere.

This system shows a more complex pattern and immediate shift of the direction of the current due to the setup of the electrodes.

Multipurpose Electrodes

A multipurpose electrode according to the present invention relates to any electrode capable of electrophysiological influence of biological cells and tissues via flow of ions (ion current).

The electrodes of the present invention are multipurpose electrodes designed so each electrode can selectively serve multiple functions such as, but not limited to, as anode, cathode or ground of a stimulation circuit.

The novelty of the invention is not related to just adding a ground to the classical two electrode (anode and cathode) circuits, but is related to having three or more fully programmable multipurpose electrodes fixed to the subject's head to allow for complex stimuli protocols.

The enabling of ground is just one of the benefits of the invention described here, as the multi-functionality of the electrodes allow for programming, and continuous adjustment of multiple type of circuits between the three (or more) electrodes.

Thus, having three or more multipurpose electrodes allows for a programmable and adaptable stimuli protocol to a human subject without altering the physical setup of the device or moving of electrodes on the subjects head. With current two-electrode setups, as seen in the existing patents references in this document, this is not possible and changing the stimuli protocol requires a replacement of one or both electrodes.

Electrodes set up for a single type of usage can only direct the current in one predetermined direction. In some existing solutions, the functionality of the two electrodes in a circuit can be reversed, but this only allows for reversing the direction of the one circuit between the two electrodes. In other existing solutions, such as high-definition tDCS, both the anode and the cathode of such a single circuit might consist of several electrodes, all functioning as either part of the anode- or the cathode-stimulation simultaneously, thus not allowing for the detailed programming that is described here.

With electrodes set up for multipurpose, it is possible to instantly reverse the function of the electrodes and thereby revolt the current between e.g. two electrodes—or between one of the electrodes and a third electrode etc.

Furthermore, each electrode can be deactivated completely, detached from the system.

In one embodiment, the invention ensures that e.g. three given electrodes A, B and C can instantly shift from A (anode)-B(cathode)-C(deactivated) to A(cathode)-B (anode)-C(deactivated), then A(cathode)-B(deactivated)-C(anode) and finally B (anode)-C(cathode). As the skilled addressee recognises, the more electrodes are applied, the more complex the system becomes, and the more detailed stimulation can be applied.

One of the functionalities of the electrodes, ground, ensures that a ground connection can be added to measure the difference between the anode and cathode providing a benchmark for the current between cathode and anode.

Another function of the ground option is that a ground electrode can collect any excess or errant electricity and lead it back to the circuit. Adding to the above example this allows for combinations such as A (anode)-B(cathode)-C(ground) and A(cathode)-B(ground)-C(anode) and immediate shifts between such combinations.

The multipurpose electrodes are preferably connected to a distribution unit with a single cable, and this cable is connected to a circuit board in a distribution unit comprising a series of transistors.

The unique combination of transistors allows selection of the functionality for each electrode, and instantly shift the functionality between anode, cathode, ground of deactivated.

In one embodiment, the option for changing between the functionality of an electrode (anode/cathode/ground/deactivation) is ensured by the design of the circuit board. Here, the desired current for an electrode, anode, cathode, ground or high impedance is routed via a pair of bipolar junction transistors with a common collector connected to the electrode lead. The base of the transistors is connected and enabled by the logic circuit. Each combination in the 2 bit space maps to a state in the electrode. This enables the system to dynamically pick the appropriate mode for each electrode during a session.

In another embodiment, the option for changing between the functionality of an electrode (anode/cathode/ground/deactivation) is ensured by the use of digital multiplexers (or muxs), electronic devices that select one of several analog or digital input signals and forwards the selected input into a single line.

The distribution unit is as such set up so it can selectively choose which cable that is connected to the electrical circuit, and with what polarity.

This enables the skilled addressee with the possibility to program the distribution unit to selectively activate or deactivate certain electrodes, and thereby program complex combinations of the flow of electricity in the users brain. An electrode that is not deactivated and not given a polarity will act as ground. Furthermore, it allows the user of the device to continuously change the type of stimuli dependent on the stimulation needs, without changing the configuration of the device or placement of electrodes.

The electrodes are kept in place with a clamp structure or brace structure (letter C on FIG. 1), which defines the fixed positioning of the electrodes and ensures the correct/desired placement on the head of the human subject.

Wet Electrode

In some embodiments, the electrode is a “wet-electrode” consisting of e.g. a sponge, a conductive grid and a shell.

The sponge can be e.g. 10 mm thick (wet state) circular and has 16 cm² in area. On one side, the sponge touches skin or hair on the scalp and on the other side it has contact to the conductive grid. Sponge tests are described in examples 9, 12, 13 and 14.

The purpose of the grid is to regulate and spread out the electricity going through the grid. The shell that has inlet for wires regulates itself to the angle/shape of the scalp and holds the grid and the sponge together.

In order to increase the connectivity/conductivity of the electrode, an electrically conductive fluid, such as a saline solution may be used to soak the sponge prior to use. This electrically conductive fluid ensures a conductive contact with the skin or hair and an equal distribution of current on the surface of the head. Analyses of the consistency of the saline solution are described in examples 6, 9, and 16.

In one embodiment, the combination of the grid, the shell, the sponge and the electrically conductive fluid ensures an even distribution of the electricity across the whole surface of the electrode, as described in Example 2.

Reusable Cellulose Sponge

The sponge material is optimized for saturated skin contact for the duration of a stimulation. Silicone rubber electrodes minimize the electrochemical polarization effects. The electrodes ensure a comfortable and consistent treatment on every use.

In one embodiment, the wet electrode is covered by a cellulose sponge, preferably a TFC sponge, as shown in Example 12, 13 and 17. In another embodiment, the sponge is a TCT sponge, as analysed in Examples 13 and 14.

In one embodiment, the wet electrode has a reusable cellulose sponge surface, such as but not limited to a TFC sponge, so the electrode surface in contact with the skin of the user can be used for multiple TES sessions before replacement.

In existing wet electrodes, repeated use of the same sponge can influence the functionality, conductivity and/or the hygiene of the sponge, due to a) the sponge surface cracking after repeated use, b) rust or similar chemical components in the electrode entering the sponge, or c) development of bacteria in the sponge and thus the electrode.

The cellulose sponges of the present invention was capable of enduring at least 20 sessions without sign of breaking or developments of cracks in the sponge surface, see Example 12.

Thus, in one embodiment, the present invention relates to a transcranial device as described herein having reusable cellulose sponge on the electrode(s).

Electrically Conductive Fluid

In one embodiment, the electrically conductive fluid may be a saline solution. The solution consists for example of a combination of water and sodium chloride, NaCl, with a density of 0.1M NaCl (≈0.5844 gram of salt to 100 millilitre of water). The saline solution is applied to the electrode sponge until the sponge is fully saturated. In one embodiment, the size of the electrode sponge is 20 square centimetres times 5-millimetre thickness and 2.67 millilitres of saline was used to saturate the sponge for a 30 minute session of transcranial electrical stimulation.

As described in Example 16, using only tap water and no salt at the electrodes is not sufficient as the electrodes need a certain level of salt to ensure connection to the skin of the subject. In one embodiment, the saline is mixed in the sponge, so the salt and the water are both added to the electrode and not mixed prior to adding. According to Example 18, the calculated minimum for the salt level needed is 0.022 gram to 100 millilitre of water. There is no upper limit for salt level in the saline solution in terms of ensuring the functionality, but the maximum concentration of salt in water used for the present purpose is 35.6 gram pr. 100 ml., however, the functionality of the electrode only dependent on minimum of salt on the electrodes.

In one embodiment, the saline solution consists of distilled water with added NaCl.

Dry Electrode

In some embodiments, the electrode is a dry electrode.

Dry electrode has the advantages of no need for skin preparation or conductive paste, potential for reduced sensitivity to motion artefacts and an enhanced signal-to-noise ratio.

The electrodes of the present invention are designed so they can interchangeably both stimulate the brain by applying current (anodal or cathodal), and record existing electrical activity in the brain (as in classical electroencephalography (EEG)). Classical electroencephalography (EEG) in the present context being an electrophysiological monitoring method to record electrical activity of the brain.

Thus, the electrodes of the present invention can measure voltage fluctuations resulting from ionic current within the neurons of the brain.

The electrodes of the present invention can thus make a recording of the brain's spontaneous electrical activity over a period, as recorded from one or multiple electrodes placed on the scalp.

The electrodes of the present invention can like EEG electrodes detect the electric signals in the brain areas directly under or nearly under the electrodes; this enables observation of the brain activity in these areas.

In one embodiment, the electrode surface is divided in two physically separate surfaces.

In a presently preferred embodiment, the electrode enabled for both EEG and neurostimulation is divided in two physically separated surfaces having one smaller surface in the center of the electrode (typically a diameter of 23 millimeter) and a surrounding annulus (typically an inner diameter of 24 millimeter, and an outer diameter of 62 millimeter).

In one embodiment, both surfaces are connected to the distribution unit with a single cable.

Typically, the center surface is used for the electroencephalographic reading, while the surrounding outer surface is used for the stimulation. However, to increase the amount of stimulation the inner surface can also be activated for stimulation (anodal, cathodal and/or ground) in combination with the surrounding surface so the whole electrode surface (typically a diameter of 62 millimetre) is active for stimulation.

The division of the electrode surface is done because the surface needed for EEG is smaller than that needed for stimulation, meaning that the EEG electrode should have a smaller surface area than that of the neurostimulation electrode.

In one preferred embodiment of the invention, the surfaces for the two electrode functionalities (EEG and neurostimulation) are placed so they share centre point.

This is necessary to ensure that the EEG electrode surface is reading the same brain area that it stimulates with the neurostimulation surface.

If, for instance, the two electrode surfaces were placed next to each other instead, but not with a shared centre point, it is not possible to use both EEG and neurostimulation for the same brain area without physically relocating the electrodes.

System for Applying a Weak Current

The present invention relates to a system for applying a weak electrical current to a human brain and/or nervous tissue, wherein the system comprises
a) at least three multipurpose electrodes applied on—or close to—the skin on the head of a human subject, wherein each electrode is wired to interchangeably function as anodal stimulation, cathodal stimulation, deactivation, and/or ground,
b) a distribution unit configured to provide the anodal stimulation, cathodal stimulation, and/or ground, and

wherein at least two of the electrodes are disposed on the right hemisphere and at least one is disposed on the left hemisphere.

In one embodiment, the multipurpose electrodes can be detached from the electrical circuit without removing them from the head of the user. If an electrode is not detached from the circuit, it might interfere with the current sent between other electrodes. For example, if electrodes A (anode) and B(cathode) are activated to create a current between the two, a third electrode C, which is in contact with the user's head and connected to the surface might interfere with the flow of current between A and B and thereby interfere with the planned flow of current. In this case, C technically acts as ground and can lead the current back to the circuit. To be able to precisely control the current flowing from A to B it is thus necessary to disconnect C and other electrodes from the circuit as long as they are in contact with the user's head.

Thus, the benefit is that it is possible to place all electrodes to the head of the user at the beginning of the stimulation, not only the ones active in the first part of the stimulation. It thereby enables quick changes of currents without physically removing electrodes not involved in the desired flow of current, from the head of the user. If the electrodes are to be removed from the head of the user for deactivation, it limits how rapid a shift in current can be.

System with a Wearable and Control Unit

In one embodiment, the system for applying the weak electrical current comprises two physical units
a) a wearable device consisting of a clamp structure holding the electrodes and the distribution unit, and
b) a control unit, detached from the wearable device.

An example of the wearable device can be seen in FIG. 1 marked with letter D, and is furthermore pictured as mounted on a human head in FIG. 2, FIG. 3, FIG. 4, FIG. 5, FIG. 6 and FIG. 7.

In FIG. 1 the clamp structure is marked with letter C, the distribution unit with letter A, and the control unit with letter B. The electrodes are pictured in FIG. 1 with numbers 1, 2, 3, 4, 5, and 6.

The wearable device according to the present invention is adapted to fit on the head of a human, as is shown in FIG. 2, FIG. 3, FIG. 4, FIG. 5, FIG. 6, and FIG. 7.

The actual wearable part of the system (FIG. 1, letter D) is designed to place the electrodes in the desired areas and keep them fixed in those positions during the whole length of the stimulation.

The wearable device is constructed of parts that make it light and flexible enough to wear for any size of adult head.

Wearable: Dock for Electricity

In one embodiment, the wearable device is designed in such a way that it is impossible to place it on the head of a user, while the device is physically connected to other devices such as, but not limited to, a charger or a computer.

This is achieved by placing the ports, and thereby the cable outlet, on the inner side of the distribution unit, and the size of the connectors make it impossible to fit the device on the head while it is connected. This is done for safety reasons, so that a user cannot wear the device, while it is connected to any power source more powerful than that of the distribution device. Thus, limiting the chance of an accident where there is a current sent to the electrodes that exceed the set maximum limits for the intensity of the electrical current that should be applied to a human brain.

In combination with the control unit, the wearable device makes up an apparatus, enabling a user to control the flow of current through the brain.

Combined, the wearable part and the control unit make a system for neuromodulation, and the system gives the user of the system the ability to achieve a predefined cognitive effect, through neurostimulation that is directed through the subject's brain.

Neurostimulation

Neurostimulation is an activation of a given part of the brain or nervous system using electrodes which apply a weak electrical current to the brain.

The Brain and/or Nervous Tissue

The present invention relates to a system for stimulation of a human brain through a weak electrical current.

In the present context, the term “brain” relates to the main organ of the human nervous system. It is located in the head, protected by the thick bones of the skull, suspended in cerebrospinal fluid, and isolated from the bloodstream by the blood-brain barrier.

The human brain is composed of neurons, glial cells and blood vessels. A neuron is an electrically excitable cell that processes and transmits information through electrical and chemical signals.

The presented system is modulating the electrical function of the neurons through creating an electrical circuit between two electrodes placed on the outside of the skin.

The present invention also relates to a system for stimulation of human nervous tissue through a weak electrical current.

In the present context, the term “human nervous tissue” relates tissue that is made up of different types of nerve cells, all of which have an axon, the long stem-like part of the cell that sends action potential signals to the next cell.

In a presently preferred embodiment, the “human nervous tissue” is placed in the skull of a human being.

Non-Invasive

There exist two main methods for neurostimulation: invasive and non-invasive methods. In general medical terms, “invasive methods” refer to puncture or incision of the skin or insertion of an instrument or injection of foreign material into the body.

The present invention does not puncture the skin, and is as such non-invasive in general medical terms.

Low-Intensity Stimulation

Within both invasive and non-invasive neurostimulation, there are two main types of stimulation: high-intensity and low-intensity stimulation. The difference between the two lies in the intensity of the stimulation: the high-intensity techniques utilize an intensity that has enough energy to force a response in the brain, while the low-intensity techniques have an intensity that can only change the threshold for how easy accessible certain areas of the brain will be during and immediately after stimulation.

In non-invasive, high-intensity neurostimulation, such as electroconvulsive therapy (or electroshock therapy) normally utilizes about 800 milliampere and has up to several hundred voltages, and the current flows for between 1 and 6 seconds.

Low-intensity stimulation such as tDCS normally utilizes less than 3 milliampere at 9 voltage, with a current flow for 20 minutes.

The present invention relates to non-invasive low-intensity stimulation.

In one embodiment, the non-invasive low-intensity stimulation is less than 20 voltage and less than 3 milliampere for various time intervals of less than 30 minutes.

The non-invasive low-intensity stimulation of the present invention is less than 20 voltage, such as but not limited to less than 19 voltage, less than 18 voltage, less than 17 voltage, less than 16 voltage, less than 15 voltage, less than 14 voltage, less than 13 voltage, less than 12 voltage, less than 11 voltage, less than 10 voltage, less than 9 voltage, less than 8 voltage, less than 7 voltage, less than 6 voltage, less than 5 voltage, less than 4 voltage, less than 3 voltage, less than 2 voltage, and/or less than 1 voltage.

The non-invasive, low-intensity neurostimulation of the present invention is utilizing a mild effect which does not hold enough power to force an effect in the brain.

The low-intensity stimulation only adds a little extra energy in the natural processes in the brain, adding extra to the naturally ongoing electrical processes in the brain and thereby modulating the accessibility and activation/deactivation of the targeted brain areas.

The system of the present invention preferably provides low-intensity neurostimulation.

Transcranial Electrical Stimulation (TES)

Low-intensity neurostimulation, neuromodulation and/or transcranial electrical stimulation (TES) applies very low amounts of electrical stimuli/current to the scalp of a human subject utilizing electrodes.

There are many forms of non-invasive, low-intensity neuromodulation techniques, but a common version is transcranial electrical stimulation (TES), where the simplest form is transcranial Direct Current Stimulation (tDCS), but more complex versions of transcranial electrical stimulation (e.g. tACS, tRNS, tPCS) can also be applied. The main difference between the techniques is the details in the type of electricity utilized, while the intensity is similar in all the non-invasive, low-intensity weak electrical current neuromodulation techniques.

In one embodiment, the present invention relates to a non-invasive, low-intensity neuromodulation technique selected from the group consisting of tDCS, tACS, tRNS, tPCS.

The weak current emulates the natural electrical activity in the brain, which is determining which areas are activated and which are deactivated. However, due to the low intensity of the current, it does not hold the power to activate or deactivate. It supports the natural activity in the brain in such a way that it requires less natural electrical activity to activate or deactivate the areas where the current is directed. A simple way to view it is that it changes the threshold for how much natural activity is needed to activate a given area of the brain.

According to the polarity of the two electrodes, the current can be directed in a given direction from anode to cathode through the brain. This means that the placement of the electrodes dictate in what direction the current will travel through the brain, and thereby which areas of the brain will be affected.

The polarity of the electrodes thereby dictate in which direction the current flows, as it will flow from the anode to the cathode. The result of the direction of the current is that the area closest to the anode will get a positive charge, and the area closest to the cathode will get a negative charge.

When positively charged stimulation (V+) is delivered, the electrical current causes a depolarization of the resting membrane potential, which increases neuronal excitability and allows for more spontaneous cell firing. When negative stimulation (V−) is delivered, the current causes a hyperpolarization of the resting membrane potential.

This decreases neuron excitability due to the decreased spontaneous cell firing. This leads to the opposite effects in the two areas: area (V+) by the anode gets a lower threshold, and the area (V−) by the cathode gets a higher threshold. The result of the modified threshold is that it takes less natural electrical activity to activate area (V+), and more natural activity to activate area (V−).

Numerous studies verify that low-intensity transcranial electrical stimulation is safe for use in humans and that it is linked with only rare and relatively minor adverse effects.

Weak Electrical Current

Numerous studies verify that low-intensity transcranial electrical stimulation (TES) is safe for use in humans, and that it is linked with only rare and relatively minor adverse side effects. TEM delivered at a level of less than 2 mA and administered according to stimulation guidelines has been shown to be safe for use in both healthy volunteers and patients with neurological injury.

Using a rat model, researchers investigated the safety limits of one version of TEM, transcranial electrical stimulation (tDCS), with extended cathodal tDCS and found the charge density threshold to be at least two orders of magnitude greater than the charge currently administered in humans.

Thus, a weak electrical current according to the present invention relates to an electrical current of less than 3 milliampere (mA) per electrode, such as but not limited to less than 2.75 mA per electrode, less than 2.5 mA per electrode, less than 2.25 mA per electrode, less than 2 mA per electrode, less than 1.75 mA per electrode, less than 1.5 mA per electrode, less than 1.25 mA per electrode, less than 1 mA per electrode, less than 0.75 mA per electrode, less than 0.5 mA per electrode, or less than 0.25 mA per electrode.

In addition to the current per electrode, the amount of current per square millimetre is crucial, as a too high density per square millimetre can be painful or even burn the skin of the subject.

The density is defined by the combination of electrode surface and current per electrode. In one preferred embodiment, the presented invention the electrodes have a diameter of e.g. 45 millimetre, giving a surface density of 62.5 microampere at 1 milliampere.

Sham

Sham stimulation is used as a control. Sham stimulation emits a brief current, but then remains off for the remainder of the stimulation time. With sham stimulation, the person receiving the weak electrical current does not know that they are not receiving prolonged stimulation. By comparing the results in subjects exposed to sham stimulation with the results of subjects exposed to actual stimulation, researchers can see how much of an effect is caused by the current stimulation, rather than by the placebo effect.

tDCS

In one embodiment, the weak electrical current is direct current stimulation, such as tDCS or similar.

Transcranial direct current stimulation (tDCS) is a form of neurostimulation, which uses constant, low current delivered to the brain area of interest via electrodes on the scalp.

In a preferred embodiment, tDCS relates to small direct constant current at 0.5-2 mA.

tACS

Transcranial alternating current stimulation (tACS) is also a non-invasive means by which alternating currents applied through the skull over the occipital cortex of the brain entrains in a frequency-specific fashion the neural oscillations of the underlying brain

The weak electrical current of the present invention also relates to bidirectional, biphasic current in sinusoidal waves with 0.25-1 mA.

The preferred frequency may be 1, 10, 15, 30, and 45 Hz, and voltage of 5-15 mV.

tPCS

The weak electrical current of the present invention also relates to unidirectional, monophasic current pulses in typically rectangular waves; can be bidirectional/biphasic

The preferred average intensity 0.6-1 mA, and frequency 1 Hz-167 kHz

tRNA

The weak electrical current of the present invention also relates to an alternate current along with random amplitude and frequency between 0.1 and 640 Hz.

The preferred intensity is between −500 and +500 μA with a sampling rate of 1280 samples providing a current of 1 mA.

Increasing Intensity

In one embodiment, the current is delivered to the electrodes with an increasing intensity, starting at 0 and building up to the full intensity over time.

The benefit of this increase in intensity is two-folded: it both makes the application of current less painful for the subject and it makes the stimuli less abrupt for the brain.

In one embodiment, the increase is 0.001 mA per second starting at 0.001 at time 0.

Thus, in 2 mA stimulation, the current reaches maximum after 210 seconds.

Physically Detached

The present invention relates to a system that can ensure a rapid switch in the flow, and multiple simultaneous flows and opposite flows through the multipurpose electrodes (anode, cathode, ground). Furthermore, electrodes can be physically detached from the electrical circuit, and thereby deactivated without requiring a physical rewiring of the electrodes.

In some embodiments, the electrodes may be detached completely from the circuit without being physical removed from the head of the subject.

Switch of Flow

The present invention further relates to complex flows involving a continuous switch of flow between electrodes, and combinations of various types of currents (e.g. tDCS, tACS, tRNS, tPCS).

The present inventors disclose why it is desired to instantly switch the neuromodulation from one area of the brain to another; by changing the functionality of a number of electrodes all mounted the head of a subject.

The research presented in the present application demonstrates how different type of stimulation is needed for supporting different cognitive tasks, and this type of research can predefine how and when such switches should happen. This type of flexible neurostimulation is only made possible by the multipurpose electrodes of the present invention, which can target a wide range of cognitive functions.

The examples disclosed herein demonstrate that depending on which type of creative problem solving the user is engaged in, various brain regions or areas must be selectively stimulated, and as the nature of the task changes, the stimulation has to change accordingly. Therefore, it is necessary with at least three electrodes constantly applied at the scalp of the user.

The number of electrodes applied depends on the complexity of the change.

In a presently preferred embodiment, the invention relates to at least six electrodes.

These electrodes each target different areas involved in creative problem solving, and then the various electrodes can be activated (anode/cathode/ground) for stimulation or deactivated. These switches between electrode activation/deactivation have to be instant. The combination of activation/deactivation of the electrodes creates a predetermined current in the subject's brain and targets the desired brain areas.

The Distribution Unit

The distribution unit can provide the weak electrical current per electrode as anodal stimulation, cathodal stimulation, and/or ground, or completely deactivate electrodes by detaching them from the electrical circuit. These functionalities have been tested in Example 2.

The distribution unit comprises means for receiving electrical signals from each electrode.

The distribution unit can sense the drop in current, and thereby calculate the resistance between electrodes and report to the control unit if electrodes are wrongly placed.

In this way, the distribution unit can measure the fall in current between the electrodes, measure the resistance between the two and thereby report to the control unit, whether the current is delivered in the desired manner. This functionality has been tested in Example 2.

The distribution unit may comprise a battery and a circuit board, wherein the battery delivers the electrical current to the electrodes through the circuit board.

Optionally the distribution unit may include a wireless communication device such as but not limited to a Bluetooth device, which receives signals from the control unit and instructs the distribution device how to direct the current.

Receiving Electrical Signals

In addition to direct current to the various electrodes based on the signals received from the control unit, the distribution unit may also be configured for receiving electrical signals from each electrode and return these as a digital signal to the control unit

This feature allows for also using the electrodes to passively read the natural electrical activity in the brain of the subject and report this activity to the control unit. This is made possible by the use of EEG-activated electrodes.

This feature can be used to measure, whether the electrodes are in the correct position, and for configuration of the headset to individual difference in brain activity.

It is also used to passively read the brain activity of the subject, as in classical EEG, so this activity can be stored in the control unit.

This recorded and stored activity can later be induced again into the subject, using the system's neurostimulation ability.

This allows the subject to put on the wearable part of the system to record a given brain activity, and this activity is stored in the control unit.

At a later point, the user can put on the wearable part of the system and use the control unit to select any previously recorded activity, and the system will use the stored signals to seek to recreate the same activity in the subject's head using a weak electrical current.

The Control Unit

The control unit comprises means for communication with the distribution unit and thereby the wearable device.

In one embodiment, the means for communication with the distribution unit is wireless. An example of such a system can be seen in FIG. 1, where the control unit is letter B.

The control unit contains variable predefined sequence of electrode activation (anode/cathode/ground) and/or deactivation, and communicates these sequences to the distribution unit that is then activating/deactivating the desired electrodes accordingly. Each of these sequences are stored in the control unit as programs that can be activated to initiate the neurostimulation.

Operation of the control unit may be done directly by the human subject (the end user), by other users than the subject wearing the wearable device, or trained staff to for example activate a predefined program stored in the control unit or to record a naturally brain activity. The control unit may also be operated remotely through digital communication.

The control unit comprise a user interface communicating with the distribution unit. The communication can be wireless or linked, and in one embodiment, the control unit can for example be a smart phone, or operated by a smart phone or other standard digital device such as a computer.

In one embodiment, the control unit provides predetermined signal(s) in the form of programs to the distribution unit.

Activate a Predefined Program

The control unit has stored a number of programs, each containing predefined stimulation sequences defining a certain activation/deactivation pattern for electrodes, which the operator can choose. When the user selects a given program, the control unit communicates with the distribution unit, sending a signal instructing the distribution unit to activate the predefined stimulation sequence, consisting of electrode activation (anode/cathode/ground) or deactivation. These programs can also be flexible, so the user or operator can modify and adjust programs during stimulation.

In one embodiment, a predefined stimulation sequence is a program consisting of digital codes.

In a presently preferred embodiment, a program consists of one or more strings, each containing information about 1) stimulation duration (of a sequence, in seconds), 2) intensity (of the neurostimulation, in milliampere), and 3) the activation for each of the electrodes (anode(a)/cathode(c)/ground(g)/deactivated(0)).

The three elements and all electrodes are separated by “:”, and each string starting with “[” and ending with “]”. In example, if the neurostimulation contains 600 seconds of 2 mA stimulation with electrode A as cathode and B as anode, and the two remaining electrodes C, D, E, and F deactivated, the string will be [600:2:Ac:Ba:C0:D0:E0:F0]. This is an example of a string. A program consists of at least one such string. In a more complex program there are multiple strings. One example of a program with four strings is:
[250:1,8:Ac:Ba:C0:D0:E0:F0]
[360:2,2:Ac:Bg:C0:D0:Ea:F0]
[500:2:A0:B0:Cg:D0:Ea:Fc]
[360:2,5:Aa:B0:Cg:Dc:E0:F0]

In another example, the control unit has a program UP, defining a 10 minute 2 mA activation of electrodes A (anode), D (cathode), E (ground), and all other electrodes deactivated; followed by a 5 minute 2.3 mA activation of electrodes A(cathode), B (anode), all other electrodes deactivated; followed by a 5 minute 2 mA activation of electrodes B (anode), F (cathode), all other electrodes deactivated. The program UP would then consist of the following three strings:

[600:2:Aa:B0:C0:Dc:Eg:F0]

[300:2,3:Ac:Ba:C0:D0:E0:F0]

[300:2:A0:Ba:C0:D0:E0:Fc]

Record Brain Activity

The control unit comprises means for detecting electrical signals from the subject's brain or nervous tissue and digitalizes the recorded signal(s). The control unit can both record these electrical signals locally and/or forward the data to another device e.g. stream the date to a cloud service.

When the subject wants to record a naturally brain activity, the control unit is able to receive a digital signal from the distribution unit.

The brain activity transmitted to the control unit from the distribution device contains information about the contemporary brain activity of the subject wearing the wearable device. This data is used for three purposes: a) placement, b) calibration, and c) creating new programs.

Placement

The recorded signals are used to analyze whether the wearable device is correctly placed, so all electrodes are in the right position on the head.

In one embodiment, this is done by instructing the subject to solve predefined tasks while wearing the wearable device. These tasks can be physical (“move your arms”) or cognitive (“think of a positive memory” or “what is 241*32 divided by 3.5”), and can be given to the subject verbally, on paper or on a screen.

For each of the tasks, there is an expected type of brain activity associated with solving the task. As the subject solves the tasks, the distribution records the brain activity during each of the tasks using the EEG electrodes, and sends the recorded brain activity to the control unit.

In the control unit, the received recordings are compared with the expected brain activity for each of the tasks. If the recorded activity for each of the tasks does not match the expected brain activity, it means that the device is not correctly placed.

For each of the tasks, a certain type of activity is expected under each of the electrodes. Thus, dependent on which electrode has recorded an unexpected activity, it is possible to detect which of the electrodes is not correctly placed.

Based on the result of the comparison of the data, the user is informed of whether the wearable device is correctly placed, and of which electrodes that are not correctly placed. After adjusting the placement of the electrodes, the process may be repeated to assess whether the adjustment was correct.

In one embodiment of the system, the analysis, where the recorded data compared to expected brain activity is performed elsewhere, the control unit sends the data to a centralized server for analysis and receives the output from the analysis.

Calibration

In one embodiment, the recorded signals are also used to calibrate the device to individual users. Every person has an individual way of using his/her brain, and these differences in brain usage have implications for the effectiveness of the neuromodulation. Therefore, it is desirable to investigate the exact individual brain patterns of each user, so the various programs can be calibrated accordingly.

The data used for such calibration is collected in the same way as when testing for correct placement, but the tasks given are different as they have the purpose of recording the brain activity involved in creative problem solving.

In comparing the recorded activity to the expected activity in the brain areas under each electrode, it is possible to assess whether the user has an abnormally high or low activity in the brain areas targeted with neuromodulation.

As an example, a given user might have abnormally high activity in the brain area under electrode A and abnormally low activity under electrode E. Thus, for the particular user any program utilizing electrode A should use a lower intensity of the stimulation of electrode A, and any program utilizing electrode E should have a higher intensity for electrode E.

Creating New Programs

Another consequence of the individual differences in brain activity is that some users might have a radically different way of utilizing the brain for creative problem solving. Thus, for some users the predefined programs might not achieve the desired effect.

In one embodiment, the system of the present invention is capable of creating new stimulation programs, wherein such a configuration or settings is obtained from the recorded digital data and is returned to the control unit

The system of the present invention comprises means for analysing the recorded digital data. The system may also comprise a configuration or settings obtained from the analysing means, which is returned to the control unit.

In example, a singular user might not, contrary to the findings from the general research presented here, use the brain area under electrode A for individual creativity, but rather utilize the brain area under electrode B unlike most other users.

Thus, a program using electrode A for individual creativity may not work optimal for said user, as electrode B should be used instead. In the ‘creating new programs’ mode, the user is given the option to record a specific type of brain activity, and the pattern recorded will be stored in the control unit. If the user e.g. has an experience of a very effective individual creativity process, the user can use the wearable device to record the brain activity during this effective process.

In its simplest form, this signal could show that the user has a strong activation of the brain areas under electrodes B and D, and a strong deactivation of the brain areas under electrode A. In the control unit, this recording is stored as a new program for individual creativity. If the user, at a later point, would like to work with individual creativity, the brain activity previously recorded would be sought reproduced using neuromodulation. In the given example, the user's individual program for individual creativity would involve an anodal stimulus in electrodes B and D, and cathodal stimuli in electrode A. Thus, the program could for example be:

[300:2,4:Ac:Ba:C0:D0:E0:F0]

[300:2,4:Ac:B0:C0:Da:E0:F0]

[300:2,4:Ac:Ba:C0:D0:E0:F0]

[300:2,4:Ac:B0:C0:Da:E0:F0]

According to the present invention, the possibility to create new programs is utilized to record and reproduce brain activity involved in the creative problem solving process, but the device itself can also record any brain activity involving the brain areas under the electrodes. In other embodiments of the invention, any brain activity involving the areas under the electrodes can be recorded and reproduced.

Types of Stimulation

Based on the signal sent from the control unit to the distribution unit, the distribution unit activates (anode/cathode/ground) or reactivates the electrodes as defined in the signal. The signal also contains the time for each activation/deactivation for each electrode.

Anodal

The anodal activation of an electrode is an electrically positively charged (V+) stimulation that increases the neuronal excitability of the area being stimulated. In this document an area receiving positive stimuli is referred to as “V+[BrainArea]”

Cathodal

The cathodal stimulation is an electrically negatively charged (V−) stimulation decreases the neuronal excitability of the area being stimulated. In this document an area receiving negative stimuli is referred to as “V−[BrainArea]”

Ground

In the current context ground (Vg) can have two functions. The ground function can be used as a safety electrode, that will collect the current going from the anode (V+) if the cathode (V−) fails in receiving the current.

In a setup with three electrodes (V+, V− and Vg), the current is supposed to go from V+ to V−, but if for some reason V− fails the current will be directed to Vg that then acts as V−. The purpose of Vg is to ensure a closed circuit if V− fails, and the placement of Vg gives the opportunity to control where the current travels. Vg is therefore normally either placed close to V− or in a place where it is considered harmless to collect the current.

The second function of Vg is as a reference point in an electrical circuit, from which voltages are measured, and more complex types of currents sent between V+ and V− will use Vg as the reference point for calculating the current in V+ and V−.

One example of such ‘more complex’ currents is transcranial alternating current stimulation (tACS), where the current is alternating over V+ and V− using Vg as the reference point.

Deactivation

In addition to anodal, cathodal or ground activation of the electrodes, all electrodes can be deactivated, so they are physically detached from the electrical curcuit.

This is a necessary functionality, as the current induced in the brain will go to the closest active electrode, thus with multiple electrodes simultaneously in contact with the user's head, where only some of them active in stimulation, it is necessary to be able to deactivate electrodes, so they do not pick up the current and thereby disturb the desired neuromodulation.

Time of Stimulation

Dependent on the desired cognitive effect, various time of stimulation can be used, varying from milliseconds to hours.

In one embodiment, the total stimulation time may by 1-60 minutes, such as but not limited to 1-45 minutes, 1-30 minutes, 1-20 minutes, 1-15 minutes, 1-10 minutes or 1-5 minutes per area.

The electrical stimulus is applied to the brain for a short time period, normally between 10 and 20 minutes, which is the time needed to change the threshold sufficiently.

One of the aspects of neurostimulation is its ability to achieve cortical changes even after the stimulation is ended. The duration of this change depends on the length of stimulation as well as the intensity of stimulation. The effects of stimulation increase as the duration of stimulation increases, or the strength of the current increases.

Typically, the stimulation time is minutes, such as but not limited to 5 minute sequences (defined as ‘a string’ in the programming), each sequence defining a given activation (anode/cathode/ground) or deactivation of electrodes.

One neurostimulation program can consist of one or multiple sequences, typically in a total maximum of, but not limited to, 4 sequences resulting in a total stimuli time of 20 minutes.

The skilled addressee would acknowledge that the stimulation time and placement of the electrodes depends on the desired cognitive effect.

In one embodiment of the invention, a combination can furthermore consist of several different types of electrode activation. In such embodiments, for instance a 5-minute sequence contains a more elaborate description for activation/deactivation of electrodes.

One example is the use of transcranial alternating current stimulation (tACS), where the current is alternating between two or more electrodes with one shared or multiple different ground electrodes. In such an embodiment, the 5-minute sequence string will contain more elaborate information about how the various electrodes are involved in the alternating current.

Fuses

The present invention also comprises fuses at various positions e.g. where a wire meets an electrode there should for example be an ampere fuse. The purpose of using fuses is to decrease the risk of exposing the user of the device to high-intensity stimuli that can force changes in the brain or in worst-case scenario damage the skin and/or the brain of the user. If the distribution unit fails, for instance if it is accidentally short-circuited with a liquid, and the battery instantly discharges into the system, the fuses will ensure that the current does not reach the head of the subject.

Further, where a wire meets an electrode there should for example be a voltage fuse.

Ideally, each electrode has an individual ampere and voltage fuse as the last point between the electrical system and the actual electrode to prevent too high power transferred to the subject.

Also, where wires are connected to the control unit there should for example be a voltage switch.

Furthermore, an automatic voltage switch at each electrode should ideally be available.

In one embodiment, the invention has at least one fuse having an individual ampere fuse and/or voltage fuse placed between an electrode and the distribution unit.

In another embodiment, the invention relates to a system, wherein at least two fuses having an individual ampere fuse and/or voltage fuse, one being placed at the wire/electrode connection and one being placed either at the wire/distribution unit connection or incorporated into the distribution unit.

Placement

The multipurpose electrodes of the present invention may be placed on different areas on the head of the human subject, and in its simplest form, they are disposed in a fixed pattern, where at least one is placed on the right hemisphere, and at least one is disposed on the left hemisphere.

In the present context, an electrode disposed on or near the midline is accounted as disposed on the right hemisphere.

In a presently preferred embodiment, the multipurpose electrodes are placed in a specific pattern, wherein at least two electrodes are disposed at the prefrontal cortex (or over the ears) and at least one electrode is disposed in the back of the head.

Thus, in one embodiment, the multipurpose electrodes are placed in a pattern, wherein at least two of the electrodes are disposed on the frontal lobe and at least one is disposed on the partial lobe.

In one embodiment, the multipurpose electrodes are placed in a pattern, wherein at least two of the electrodes are disposed at the prefrontal cortex and at least one is disposed at the occiput.

In another embodiment, the multipurpose electrodes are placed in a pattern, wherein at least one of the electrodes is disposed on the frontal lobe and at least two are disposed on the partial lobe.

In another embodiment, the multipurpose electrodes are placed in a pattern, wherein at least three of the electrodes are disposed on the frontal lobe.

In another embodiment, the multipurpose electrodes are placed in a pattern, wherein at least three of the electrodes are disposed on the partial lobe.

In one embodiment, one or more multipurpose electrodes are placed on the body. The advantage of placing at least one electrode on the body and not the head of the subject is that it can be used as a reference point and/or a stimulation electrode. As a reference point, it can detect the skin resistance other places on the body than the head. As a stimulation electrode, it can be used to create the circuit with an electrode placed on the user's head, when it is only desired to have a single stimulation in the brain (V+ or V−).

According to the research presented below, the six areas most relevant for the product are listed in Table 4.

The International 10-20 System for Placement of EEG Electrodes

The ‘10-20 system’, ‘10-20 EEG placement scheme’ or ‘International 10-20 system’ is an internationally recognized method to describe and apply the location of scalp electrodes in the context of an EEG test or experiment. This method was developed to ensure standardized reproducibility so that a subject's studies could be compared over time and subjects could be compared to each other. This system is based on the relationship between the location of an electrode and the underlying area of cerebral cortex. The “10” and “20” refer to the fact that the actual distances between adjacent electrodes are either 10% or 20% of the total front−back or right−left distance of the skull, as seen in FIGS. 9 and 10. Each site has a letter to identify the lobe and a number to identify the hemisphere location. The letters F, T, C, P and O stand for frontal, temporal, central, parietal, and occipital lobes, respectively. (Note that there exists no central lobe; the “C” letter is used only for identification purposes.) Even numbers (2, 4, 6, 8) refer to electrode positions on the right hemisphere, whereas odd numbers (1, 3, 5, 7) refer to those on the left hemisphere. A “z” (zero) refers to an electrode placed on the midline. In addition to these combinations, the letter codes A, Pg and Fp identify the earlobes, nasopharyngeal and frontal polar sites respectively. Two anatomical landmarks are used for the essential positioning of the EEG electrodes: first, the nasion, which is the distinctly depressed area between the eyes, just above the bridge of the nose; second, the inion, which is the lowest point of the skull from the back of the head and is normally indicated by a prominent bump. These anatomical landmarks, and their relation to the 10-20 system, is illustrated in FIG. 10.

In the present application, all electrode placements are given based on the international 10-20 system for placement of EEG electrodes unless otherwise stated.

In one embodiment, any electrode placement defined by a point in the 10-20 system refers to the electrode being closest to that point. It does not as such mean that the centre of the electrode is aligned with the precise 10-20 system coordinate. For example, an electrode referred to here as being located in T4 is not necessary in T4 exactly, but closer to T4 than any of the surrounding 10-20 system coordinates.

According to the current invention, electrodes can as such have an offset from the precise 10-20 system, but the placement of the electrode is referred to in terms of the closest 10-20 system coordinate.

It is also crucial to emphasize that the purpose of the electrode placement is to target certain predefined brain areas inside the scull. The use of the 10-20 system coordinates is a way of seeking to achieve the correct placement on the outside of the scull to target the desired brain areas.

In individual users, the 10-20 coordinates used might not map directly with the desired underlying brain areas. If, for individual, anatomical or abnormal reasons, in an individual the 10-20 placement does not match with the desired brain areas, the electrodes must be moved accordingly to match the desired brain area. In this case, the placement of electrodes will still match with the desired brain areas, even though they are not in the locations defined here in terms of the 10-20 system.

Therefore, in Table 4 all the brain areas which the invention is seeking to stimulate according to their 10-20 placement, are listed. In this list, the far-left column is the crucial, and the 10-20 coordinates are just represented as ways of seeking to achieve the correct placement of electrodes on the outside of the head of a user.

Single Placement

A list of all the electrode placements in the current invention can be found in Table 4.

Below is a detailed explanation of selection of these placements.

In one embodiment, an electrode is placed over the left ear at position T3 on the 10-20 EEG placement scheme.

The electrode is pictured as 1 in FIG. 1. The placement can be all points surrounding T3, which are not closer to any other coordinates in the 10-20 system. The purpose of the electrode in T3 is to stimulate the left Temporal Lobe.

If, due to individual differences, an electrode placed in T3 does not stimulate left Temporal Lobe, the location of the electrode has to be adjusted in order to stimulate the left Temporal Lobe.

In one embodiment, an electrode is placed over the right ear at position T4 on the 10-20 EEG placement scheme.

The electrode is pictured as 2 in FIG. 1. The placement can be all points surrounding T4, which are not closer to any other coordinates in the 10-20 system. The purpose of the electrode in T4 is to stimulate the right Temporal Lobe.

If, due to individual differences, an electrode placed in T4 does not stimulate the right Temporal Lobe, the location of the electrode has to be adjusted so to stimulate the right Temporal Lobe.

In one embodiment, an electrode is placed in the middle of the top of the back of the head at position Pz on the 10-20 EEG placement scheme.

The electrode is pictured as 3 in FIG. 1. The placement can be all points surrounding Pz, which are not closer to any other coordinates in the 10-20 system. The purpose of the electrode in Pz is to stimulate Precuneus.

If, due to individual differences, an electrode placed in Pz does not stimulate Precuneus, the location of the electrode has to be adjusted so to stimulate Precuneus.

In one embodiment, an electrode is placed at the left side of the forehead at position F3 on the 10-20 EEG placement scheme.

The electrode is pictured as 4 in FIG. 1. The placement can be all points surrounding F3, which are not closer to any other coordinates in the 10-20 system. The purpose of the electrode in F3 is to stimulate the left Dorsolateral Prefrontal Cortex. If, due to individual differences, an electrode placed in F3 does not stimulate the left Dorsolateral Prefrontal Cortex, the location of the electrode has to be adjusted so to stimulate the left Dorsolateral Prefrontal Cortex.

In one embodiment, an electrode is placed at the right side of the forehead at position F4 on the 10-20 EEG placement scheme.

The electrode is pictured as 5 in FIG. 1. The placement can be all points surrounding F4, which are not closer to any other coordinates in the 10-20 system. The purpose of the electrode in F4 is to stimulate the right Dorsolateral Prefrontal Cortex.

If, due to individual differences, an electrode placed in F4 does not stimulate the right Dorsolateral Prefrontal Cortex, the location of the electrode has to be adjusted so to stimulate the right Dorsolateral Prefrontal Cortex

In one embodiment, an electrode is placed over the right eye at position Fp2 on the 10-20 EEG placement scheme.

The electrode is pictured as 6 in FIG. 1. The placement can be all points surrounding Fp2, which are not closer to any other coordinates in the 10-20 system. The purpose of the electrode in Fp2 is to stimulate the right Orbitofrontal Cortex.

If, due to individual differences, an electrode placed in Fp2 does not stimulate the right Orbitofrontal Cortex, the location of the electrode has to be adjusted so to stimulate the right Orbitofrontal Cortex.

Combinatory Placements

A list of all the electrode placements in the current invention can be found in Table 4. The electrode placements are combined in various ways as will be described here.

In one embodiment, the multipurpose electrodes are placed in a pattern of two electrodes, wherein one electrode is placed on the left Dorsolateral Prefrontal Cortex (L DLPFC) and one on the right Dorsolateral Prefrontal Cortex (R DLPFC), locations F3 and F4 on the 10-20 EEG placement scheme. This is to be able to manipulate L DLPFC and R DLPFC to either have an increased or decreased neuronal excitability. This placement can give the alternatives of:
L DLPFC (V+) and R DLPFC (V+)
L DLPFC (V+) and R DLPFC (V−)
L DLPFC (V−) and R DLPFC (V+)
L DLPFC (V−) and R DLPFC (V−).

In one embodiment, the multipurpose electrodes are placed in a pattern of three electrodes, wherein one electrode is placed on L DLPFC (T3); one on the R DLPFC (T4), and one over Precuneus, Pz on the 10-20 EEG placement scheme.

This position is configured to target a combination of L/R LDPFC and Precuneus, as shown in table 1. This combination of electrodes function, as described in research, creates a correlated activity between L/R LDPFC and Precuneus. In this embodiment, if L and/or R DLPFC are stimulated positively (V+), so should Precuneus. And if L and/or R DLPFC are stimulated negatively (V−), so should Precuneus.

In one embodiment, the multipurpose electrodes are placed in a pattern with electrodes in T3, and/or T4, and/or Pz as described above, in combination with an electrode over the right ear on T4 in the 10-20 EEG placement scheme, targeting the right Temporal Lobe. This combination of electrodes ensures all the types of stimuli listed in Table 1.

In one embodiment, the multipurpose electrodes are placed in a pattern, wherein at least one electrode is placed over the left ear on T3 in the 10-20 EEG placement scheme, targeting the left Temporal Lobe; and/or one electrode placed over the right ear on T4 in the 10-20 EEG placement scheme, targeting the right Temporal Lobe

In another embodiment, the pattern involving T3 and/or T4 is combined with one electrode on F3 (L DLPFC) and/or one on F4 (R DLPFC). In another embodiment, the pattern involving T3 and/or T4 and/or F3 and/or F4 is in combination with one over Pz targeting Precuneus.

In one embodiment, the multipurpose electrodes are placed in a pattern, including one electrode on Fp2 in the 10-20 EEG placement scheme, targeting the right Orbitofrontal Cortex. As can be seen in Table 2, an electrode targeting the right Orbitofrontal Cortex (Fp2) should at least be combined with an electrode targeting Precuneus (Pz), but as is shown in Table 3 an electrode targeting the right Orbitofrontal Cortex can be combined with all the other placements displayed in Table 3.

All the patterns of the at least 3-6 multipurpose electrodes described herein can be combined, as will be explained in section Use of the product below and seen in Table 3.

Use of the Product

The present invention can be used for inducing a cognitive effect in a human subject by stimulating the brain using a weak electrical current applied to the surface of the head of the subject by multipurpose electrodes configured to distribute electric energy to the electrodes according to a determined sequence, pattern or signal.

In one embodiment, the general cognitive effect induced is creative thinking, in the form of ‘creative problem solving’, ‘open-ended problem solving’ or simply ‘problem solving’.

Creative Problem Solving

While the concept of ‘creative problem solving’ might be considered undefined in everyday language, it is well defined in academic research. Here it is defined as the mental process of searching for a new and novel creative solution to a problem, a solution, which is novel, original and not obvious.

There are multiple types of academic studies of creative problem solving and the associated cognitive functions in various stages in the process.

In one branch of such creativity research, ‘the neuroscience of creativity’ or simply ‘neurocreativity’, research is aimed at understanding the brain activity and brain areas associated with the cognitive processes used in creative problem solving.

The result of these studies can be used to dictate which type of brain activity in which brain areas is desired in the cognitive processes known to be relevant in a given part of the creative process.

The research presented here is part of these types of neurocreativity studies.

Divergent/Convergent Thinking

In academic studies of creativity, a distinction is usually made between two types of cognitive modes with different related brain processes.

Divergent thinking is a thought process or method used to generate creative ideas by exploring many possible solutions. It is often used in conjunction with its cognitive opposite, convergent thinking, which follows a particular set of logical steps to arrive at one solution, which in some cases is a ‘correct’ solution. By contrast, divergent thinking typically occurs in a spontaneous, free-flowing, ‘non-linear’ manner, so that many ideas are generated in an emergent cognitive fashion. In divergent thinking any possible solutions are explored in a short amount of time, and unexpected connections are drawn. After the process of divergent thinking has been completed, ideas and information are organized and structured using convergent thinking.

Steps in Creative Problem Solving

The present invention offers the user of the system to improve the cognitive processes required for in creative problem solving. Creative problem solving relates to processing a series of different steps; each considered entailing one or multiple cognitive processes needed by the subjects participating in the process. There are various steps defined by different existing studies of creativity, but some basic steps that have to be in place in an optimal process are acknowledged. The steps that are relevant for the present invention are:
1. Individual idea generation
2. Group idea generation
3. Individual idea selection
4. Group idea selection
5. Insight moments (′aha-moments' or ‘eureka moments’
6. Increased working memory

Steps 1 and 2 are part of divergent thinking, and steps 3 and 4 are part of convergent thinking. Step 5 is related to an individual cognitive effect where a number of connections in the brain are made in an instant, providing the individual with a new answer to a known problem. Step 6, increased working memory, is known to be a crucial aspect in multiple parts of the creative problem solving process, as memory functions lie at the core of a wide range of individual cognitive functions related to creativity. The various cognitive states described above rarely exist in isolation, and at different stages in the process, the user will need various cognitive functions from the above list simultaneously.

The present invention provides the user with the ability to select from the 6 different steps above, dependent on where in the creative problem solving process the user is, and which cognitive processes and thereby brain activity in given brain areas is desired. In a normal cognitive function, without the system of the present invention, a person going through a creative problem solving process has to rely on chance to have the right cognitive process for the step(s) he is in.

To use of the present invention, the user first assess where in the creative problem solving process he is, and based on that assessment, use the system to induce the desired cognitive process(es). The user activates the desired cognitive process(es) using the control unit, and the invention seeks to reproduce the brain activity associated with the selected cognitive process(es) through neurostimulation of the brain areas involved.

In one embodiment, the control unit therefore has 8 predefined settings/programs, as listed in Table 3, offering given combinations of the 6 cognitive functions listed above.

The user can then freely select a given program, or sequence of programs, dependent on the step in the creative problem solving process, and the system will induce a weak electrical current to the user's brain to induce the desired brain activity associated with the cognitive process.

In the present invention, the neurological knowledge presented is used in combination with multipurpose electrical stimulation, to selectively activate, or deactivate, the brain areas that are associated with a certain cognitive process desired at a given step in the creative problem solving process.

Stimulating brain areas is known; but the way of targeting different areas and the shifts between different types of stimulation, to replicate a series of cognitive processes, in combination with the sequence of stimulation is unique.

In summary, the device of the present invention can support the user in achieving the right cognitive processes for creative problem solving through neurostimulation. Here, creative problem solving is understood as a combination of divergent and convergent cognitive processes. The creative problem solving process is furthermore divided in 6 different steps, all represented by a specific type of cognitive process.

Each of these cognitive processes can be induced using transcranial electrical stimulation, either one at a time or multiple simultaneously, through predefined programs or patterns of electrode activation/deactivation. Due to the different types of cognitive processes, it is necessary to have a device with a number of multipurpose electrodes that can induce the desired effects based on commands from the user of the device.

At Least 3 Electrodes

The present inventors carried out studies with the purpose of investigating the specific brain activity related to known cognitive processes in creative problem solving.

The key interest was to distinguish the individual creative process from the group creative process. The studies were designed to measure improvisation, which is considered a key to understanding the essence of creativity, and measuring changes in the brain activity according to whether the improvisation is part of a collaboration (response improvisation) or strictly individual (free improvisation).

The present inventors discovered that there are two distinct parts of the brain involved in the two types of creativity, one that is related to group creativity (understood as improvisation in response to others improvisation) and another area related to individual creativity (understood as free improvisation independent of others).

The consequence of these findings is that there are two significantly different types of cognitive processes involved in creative problem solving, one related to group creativity and one related to individual creativity. The study furthermore disclosed that each of these two cognitive processes is represented by a certain distinct brain activity, in the same part of the prefrontal cortex but with the opposite type of activity. Meaning the prefrontal area of the brain that has to be activated positively (V+) in individual creativity has to be deactivated (V−) in-group creativity, and the brain area to be activated negatively (V−) in individual creativity has to be activated (V+) in-group creativity.

What this means is that a device using TES to support the cognitive processes related to creative problem solving, has to be able to replicate both cognitive processes found in the present study: One related to individual creativity (free improvisation) and one related to group creativity (response improvisation).

Since these two types of cognitive processes are located in the same brain areas but need to be stimulated with opposite polarity, it is necessary to use multipurpose electrodes to enable the user to make rapid shifts between individual- and group creativity, without having to change the location or wiring of the electrodes.

In Example 1, which shows a study of individual and group creativity, the prefrontal activity across the left and the right hemispheres was directly compared, using the lateralization index (left−right/left+right). All voxels in the Dorsolateral Prefrontal Cortex (DLPFC) area, at all levels of significance were left lateralized during group creativity and right lateralized in the individual creativity condition (FIG. 14 and FIG. 15).

As shown in FIG. 13, this occurred in such a way that when DLPFC was activated in one hemisphere it was not activated in the corresponding area of the contralateral hemisphere. The involvement of the DLPFC in creativity is of interest because the prefrontal cortex is known to integrate already highly processed information to enable even higher cognitive functions.

The shift in polarity between the activation of the right and left DLPFC respectively demonstrate that creative behaviour is not facilitated by only one side of the brain, but can within the same subjects be dominated by either hemisphere depending on the nature of the underlying task. Example 1 demonstrates that the right DLPFC is significantly more activated in a task involving individual creativity whereas the left DLPFC is more activated in a task involving group creativity.

Group Creativity (V+LDLPFC, V−RDLPFC)

A whole brain analysis revealed that only one area was significantly more activated in the group creative condition compared to control (imitation). Activity was found in the left Dorsolateral Prefrontal Cortex (DLPFC), more specific in the superior Frontal Gyrus (or “Brodmann Area 9”). See table 5, FIG. 11.

Activation in this area was highly significant with a t-value of 7.11 (p<0.000001).

We found no deactivations at the current threshold level of False Discovery Rate (FDR) <0.05 or with a more liberal level of FDR<0.2. Likewise, there were no additional activations or trends of activations at FDR<0.2.

Individual Creativity (V−RDLPFC, V+LDLPFC)

Brain areas with a significantly higher Blood Oxygenation Level Dependent (BOLD) signal in the individual creativity condition than in the control condition (main meter) are summarized in Table 6 and illustrated in FIG. 16.

In the Frontal Lobe, activations were found in the right Dorsolateral Prefrontal Cortex (DLPFC), more specifically in the middle Frontal Gyrus (Brodmann Area 9), and bilaterally in the superior Frontal Gyrus (pre-SMA/PMA), sub-gyral (SMA), and inferior Frontal Gyrus (Operculum, Brodmann Area 44).

In the Limbic Lobe, activation was found in the right Anterior Cingulate Cortex (CMA, BA 32) and bilaterally in the Anterior Cingulate Cortex (CMA, Brodmann Area 24).

Temporal Lobe activations were found bilaterally in the superior Temporal Gyrus (STG), and transverse Temporal Gyrus (auditory cortex).

In the Parietal Lobe, activations were found bilaterally in the superior Parietal Lobule (Precuneus, Brodmann Area 7). Subcortical activations were observed bilaterally in the putamen, thalamus, insula and cerebellum.

There were no significant deactivations at the current threshold of FDR<0.05 or when using a far more liberal threshold of FDR<0.2.

The areas described above are in agreement with the areas usually found to be associated with music production by other studies with the exception of right Dorsolateral Prefrontal Cortex (DLPFC), which in studies of music has exclusively been linked to creative improvisation.

The combination of the findings in experiment A and experiment B presented in Example 1 represents the need for two multipurpose electrodes, one in each of the hemispheres of the frontal cortex, to enable the user to rapidly shift between programs for individual creativity and for group creativity.

Idea Generation and the Involvement of Precuneus in Creative Ideation (V+Precuneus)

The effects of heightened activity in both the left and right Dorsolateral Prefrontal Cortex (L and R DLPFC) is moderated by the activity in the brain area known as Precuneus.

In low-creative individuals, an opposite correlation exists between activity in the DLPFC and Precuneus, meaning that activity in Precuneus goes down when activity in DLPFC goes up.

In highly creative individuals, it is the opposite, a positive correlation, meaning that activity in Precuneus goes up when activity in DLPFC goes up.

Thus, to mimic the brain activity found in the cognitive processes of highly creative people, it is necessary to stimulate Precuneus positively (V+Precuneus), when stimulating either sides of DLPFC positively (V+ in L or R DLPFC).

Precuneus can be stimulated by using at least one, but preferably multiple, electrodes. In addition to the two multipurpose electrodes needed to switch between activity in the left and right hemisphere of the DLPFC, at least one electrode over Precuneus is needed.

This electrode has to be multipurpose, as it has to be stimulating positively for idea generation (V+) and negatively for idea selection (V−), two distinct different and equally important cognitive processes involved in creative problem solving.

While 3 electrodes can, as described above, be enough to reproduce the brain activity of highly creative individuals, in the form of idea generation combined with either individual or group creativity, there are other important elements in the creative process that can be stimulated using additionally electrodes.

Benefits of 4 Electrodes

As is shown in Table 1, #3 and #4, stimulating Precuneus is associated with improved idea generation. The relationship between idea generation and another brain area known as the Temporal Lobe is also shown (see also Table 6 and FIG. 16). During idea generation, the right hemisphere part of this brain area show heightened activity. Thus, by using an electrode placed over the Temporal Lobe, stimulating this area positively (V+Temporal Lobe) together with positive stimuli of Precuneus (V+Precuneus) will mimic idea generation by highly creative individuals (Table 1, field #3).

Benefits of 5 Electrodes

Positive stimulation over the right Temporal Lobe (V+Temporal Lobe), same area as targeted for idea generation in the above 4 electrode setup, in combination with negative stimuli over the left Temporal Lobe (V−Temporal Lobe), has showed better abilities for insight problem solving (step 5 for creative problem solving outlined above). Thus, by adding a fifth electrode the results of this study can be replicated, and added to the programs available for the user of the device as listed in Table 3. This is shown in Table 2 #2.

Benefits of 6 Electrodes

While 5 electrodes, as explained above, have a benefit for insight problem solving, there is another crucial element in the creative problem solving process known as improved working memory (step 6 in the creative problem solving outlined above). Improved working memory gives the subject increased access to working with multiple pieces of information simultaneously in the creative process. Improved working memory can be achieved by positively stimulating the right Orbitofrontal Cortex. The reference of the electrode can be any of the nearby electrodes or in Precuneus. This is shown in Table 2, #1.

Switching

The four first areas described above and their activity dependent on a) whether the creative process involves individual (V−RDLPFC, V+LDLPFC) or group creativity (V+LDLPFC, V−RDLPFC), and b) whether it involves idea generation (V+Precuneus) (V+right Temporal Lobe), or idea selection (V−Precuneus).

Therefore, throughout a creative process, it is necessary to be able to increase or decrease the activity in these four areas depending of the nature of the task at hand and the type of creative outcome desired.

In the current invention, using 6 multipurpose electrodes placed in the locations described in table 4, it is based on the findings explained above possible to neurostimulate all desired areas positively or negatively to achieve all the 8 cognitive effects listed in table 3.

For example, if the user of the system of the present invention is involved in the individual creativity (meaning working by him/her self), the system should stimulate the brain in such a way that activity in the right DLPFC is increased (V+RDLPFC) and the left DLPFC is decreased (V−LDLPFC).

If the individual process involves idea generation, then Precuneus and right Temporal Lobe should be increased (V+Precuneus, V−Right Temporal Lobe), and if it involves idea selection Precuneus should be deactivated (V−Precuneus)

This can be achieved with an electrode placed directly with an anode over the areas that should be activated and a cathode over the areas that should be deactivated.

In one embodiment, the system of the present invention can be used for enhancing individual creativity by stimulating the brain in such a way that the right DLPFC is increased and left DLPFC is decreased (V+RDLPFC, V−LDLPFC).

According to the present invention, this is done by combinations of activation in the four areas, as displayed in Table 1.

Having e.g. 4 electrodes over areas LDLPFC, RDLPFC, Precuneus and Right Temporal Lobe, using multipurpose electrodes all 4 desired effects listed in Table 2 can be achieved. Through a creative process, all these 4 effects will be desired at certain point(s), and the device can by pushing a button change between these 4 effects.

Kit of Parts

One embodiment of the present invention relates to a kit comprising
a) a device comprising at least three multipurpose electrodes as disclosed here in,
b) a container comprising a 0.1 M NaCl solution
c) instructions for placement of the device and activation of a program on the control unit, and
d) optionally replacement sponges for a wet electrode.

General

It should be understood that any feature and/or aspect discussed above in connections with the device according to the invention apply by analogy to the use and cognitive effects described herein.

The following Figures and Examples are provided below to illustrate the present invention. They are intended to be illustrative and are not to be construed as limiting in any way.

### TABLES

TABLE 1
Key functions

| | | Right Dorsal | Left Dorsal | | |
|---|---|---|---|---|---|
| | | Lateral | Lateral | | Right |
| | | Prefrontal | Prefrontal | | Temporal |
| # | Desired effect | Cortex | Cortex | Precuneus | Lobe |
| 1 | Group | V− | V+ | | |
| | creativity | | | | |
| 2 | Individual | V+ | V− | | |
| | creativity | | | | |
| 3 | Idea generation | | | V+ | V+ |
| 4 | Idea selection | | | V− | |

TABLE 2
Additional functions

| | | | Right | Left | Right |
|---|---|---|---|---|---|
| | | | Temporal | Temporal | Orbitofrontal |
| # | Desired effect | Precuneus | Lobe | Lobe | Cortex |
| 1 | Working | V− | | | V+ |
| | memory | | | | |
| 2 | Insight problem | | V+ | V− | |
| | solving | | | | |

TABLE 3
Full program list

| | Right | | | | | |
|---|---|---|---|---|---|---|
| | Dorsal | Left Dorsal | | | | |
| Stage in | Lateral | Lateral | | Right | Left | Right |
| creative | Prefrontal | Prefrontal | | Temporal | Temporal | Orbitofrontal |
| process | Cortex | Cortex | Precuneus | lobe | lobe | cortex |
| Group idea | V− | V+ | V+ | V+ | | |
| generation | | | | | | |
| Group idea | V− | V+ | V+ | V+ | V− | |
| generation | | | | | | |
| with insight | | | | | | |
| problems | | | | | | |
| Group idea | V− | V+ | V− | | | |
| selection | | | | | | |
| Group idea | V− | V+ | V− | | | V+ |
| selection with | | | | | | |
| working | | | | | | |
| memory | | | | | | |
| Individual idea | V+ | V− | V+ | V+ | | |
| generation | | | | | | |
| Individual idea | V+ | V− | | V+ | V− | |
| generation | | | | | | |
| with insight | | | | | | |
| problems | | | | | | |
| Individual idea | V+ | V− | V− | | | |
| selection | | | | | | |
| Individual idea | V+ | V− | V− | | | V+ |
| selection with | | | | | | |
| working | | | | | | |
| memory | | | | | | |

TABLE 4
Location of areas

| | Coordinates in | According to | Number in |
|---|---|---|---|
| Brain area targeted | 10-20 | claim | figures |
| Left Temporal Lobe | T3 | 9 | 1 |
| Right Temporal Lobe | T4 | 10 | 2 |
| Precuneus | Pz | 11 | 3 |
| Left Dorsal Lateral Prefrontal | F3 | 12 | 4 |
| Cortex | | | |
| Right Dorsal Lateral | F4 | 13 | 5 |
| Prefrontal Cortex | | | |
| Right Orbitofrontal Cortex | Fp2 | 14 | 6 |

TABLE 5
Areas significantly more activated in the group creativity
condition than control. Stereotaxic Coordinates with t- and
Z-score values for activations in the [Answer] task
contrasted to the [Imitate] task. Only brain regions
with a significantly increased BOLD contrast signal are shown.

| Brain regions for which t >3.68 | t-value | x | y | z | Z-Score |
|---|---|---|---|---|---|
| Frontal Cortex | | | | | |
| Left | | | | | |
| Superior Frontal Gyrus (DLPFC) (8) | 7.11 | −16 | 34 | 52 | 4.97 |

Brian atlas coordinates are in millimeters along the left-right (x), anterior-posterior (y), and superior-inferior (z) axes. In parentheses after the brain area is the Brodmann area.

TABLE 6
All areas significantly more activated in the individual
creativity condition than control.
Stereotactic coordinates with t- and Z-score values for
activations in the [Improvise] task contrasted to the
[Main Meter] task. Only brain regions with a significantly
increased BOLD contrast signal are shown.

| Summary of brain regions for which t >3.68 | t-value | x | y | z | Z-Score |
|---|---|---|---|---|---|
| Frontal Cortex | | | | | |
| Right | | | | | |
| Middle Frontal Gyrus (DLPFC), (9) | 4.34 | 32 | 44 | 30 | 3.62 |
| Superior Frontal Gyrus (pre-SMA/PMA), (6) | 5.57 | 6 | 12 | 60 | 4.32 |
| Sub-Gyral (SMA), (6) | 5.65 | 16 | −4 | 56 | 4.36 |
| Inferior Frontal Gyrus (Operculum), (44) | 5.65 | 48 | 16 | 16 | 4.36 |
| Left | | | | | |
| Superior Frontal Gyrus (pre-SMA/PMA), (6) | 4.57 | −6 | 8 | 68 | 3.77 |
| Sub-Gyral (SMA), (6) | 4.59 | −12 | −2 | 60 | 3.78 |
| Precentral Gyrus (Primary Motor Area), (4) | 6.94 | −32 | −24 | 54 | 4.95 |
| Inferior Frontal Gyrus (Operculum), (44) | 4.35 | −60 | 14 | 6 | 3.63 |
| Limbic Cortex | | | | | |
| Right | | | | | |
| Anterior Cingulate Cortex (CMA), (24) | 6.63 | 12 | 0 | 48 | 4.82 |
| Anterior Cingulate Cortex (CMA), (32) | 6.32 | 6 | 6 | 42 | 4.68 |
| Left | | | | | |
| Anterior Cingulate Cortex (CMA), (24) | 6.28 | −6 | −10 | 50 | 4.66 |
| Temporal Cortex | | | | | |
| Right | | | | | |
| Superior Temporal Gyrus, (21) | 4.49 | 48 | −4 | −12 | 3.72 |
| Superior Temporal Gyrus, (22) | 4.46 | 58 | −10 | −2 | 3.70 |
| Transverse Temporal Gyrus (Auditory Cortex), (41) | 4.44 | 42 | −24 | 8 | 3.69 |
| Left | | | | | |
| Superior Temporal Gyrus, (22) | 4.93 | −62 | −40 | 14 | 3.97 |
| Transverse Temporal Gyrus (Auditory Cortex), (41) | 6.48 | −46 | −26 | 10 | 4.75 |
| Parietal Cotex | | | | | |
| Right | | | | | |
| Superior Parietal Lobule (precuneus), (7) | 4.54 | 36 | −62 | 56 | 3.75 |
| Left | | | | | |
| Superior Parietal Lobule (precuneus), (7) | 4.27 | −38 | −54 | 66 | 3.58 |
| Other regions | | | | | |
| Right | | | | | |
| Insula | 5.21 | 42 | −18 | −8 | 4.13 |
| Putamen | 4.45 | 22 | 6 | 0 | 4.36 |
| Thalamus | 3.92 | 10 | −14 | 4 | 3.36 |
| Left | | | | | |
| Insula | 5.75 | −36 | −30 | 16 | 4.41 |
| Putamen | 3.60 | −28 | −2 | −2 | 3.14 |
| Thalamus | 4.56 | −12 | −18 | 4 | 3.76 |
| Cerebellum | | | | | |
| Right | | | | | |
| Cerebellum | 4.40 | 32 | −54 | −34 | 3.66 |
| Cerebellum | 4.36 | 46 | −52 | −40 | 3.64 |
| Left | | | | | |
| Cerebellum | 5.29 | −36 | −56 | −36 | 4.17 |
| Cerebellum | 5.62 | −42 | −58 | −48 | 4.34 |

Brian atlas coordinates are in millimeters along the left-right (x), anterior-posterior (y), and superior-inferior (z) axes. In parentheses after each brain area is the Brodmann area.

### BRIEF DESCRIPTION OF THE FIGURES

FIG. 1 shows one possible design for the device. Objects 1-6 are six multipurpose electrodes and object A is the distribution unit. Object B is the control unit here wirelessly communicating with the distribution unit (A). The brace structure supporting the electrodes, containing the wiring and the distribution unit, is marked with the letter C.

The wearable part of the system, consisting of the brace structure (C), electrodes (1-6), and the distribution unit (A) is marked with the letter D.

Electrodes 2, 5 and 6 are in the right hemisphere, and electrodes 1 and 4 are in the left hemisphere. Electrode 3 is placed on the median line between the two hemispheres. The position of objects 1-6 is predetermined to target the areas for example described in table 4:
1 is targeting the Left Temporal lobe through a position over T3 in the international 10-20 EEG coordinate system
2 is targeting the Right Temporal lobe through a position over T4 in the international 10-20 EEG coordinate system
3 is targeting the Precuneus through a position over Pz in the international 10-20 EEG coordinate system
4 is targeting the Left Dorsal Lateral Prefrontal Cortex through a position over F3 in the international 10-20 EEG coordinate system
5 is targeting the Right Dorsal Lateral Prefrontal Cortex through a position over F4 in the international 10-20 EEG coordinate system
6 is targeting the Lateral Orbitofrontal cortex through a position over Fp2 in the international 10-20 EEG coordinate system.

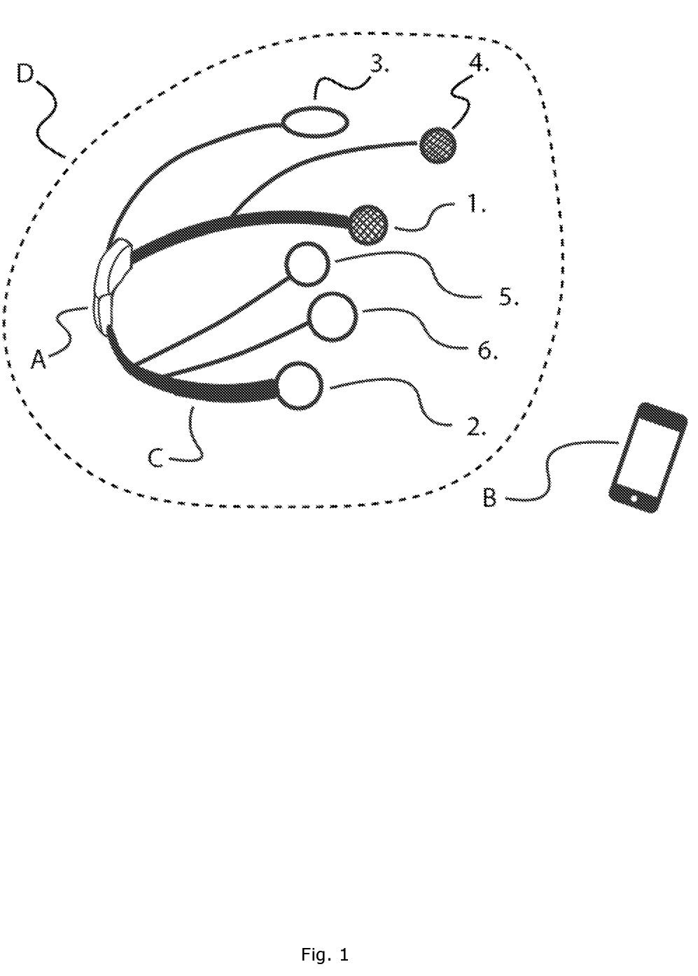

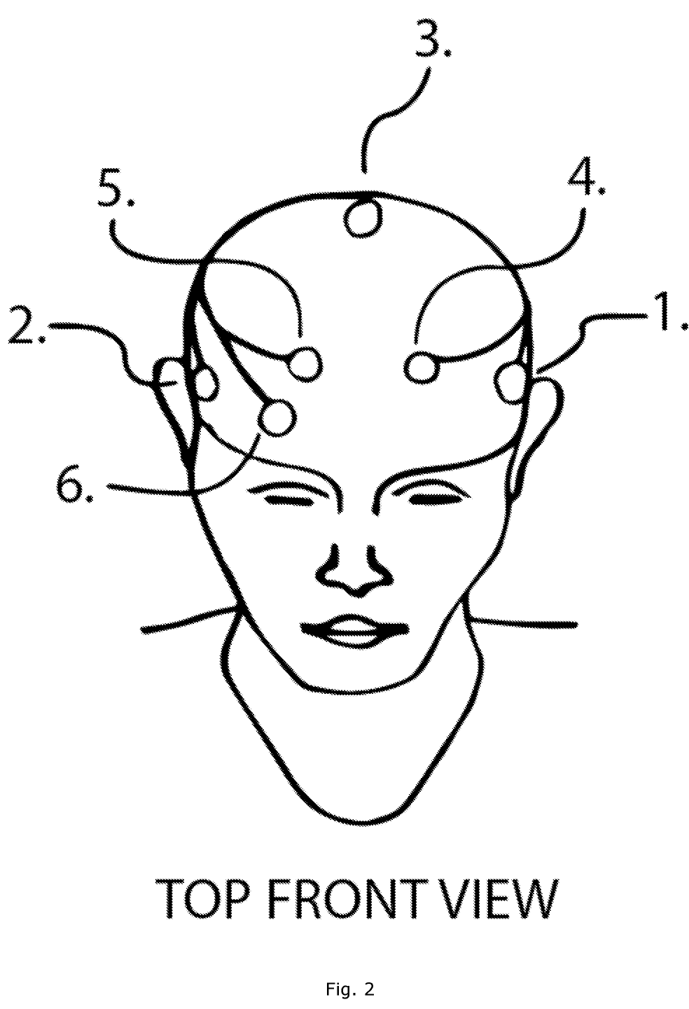

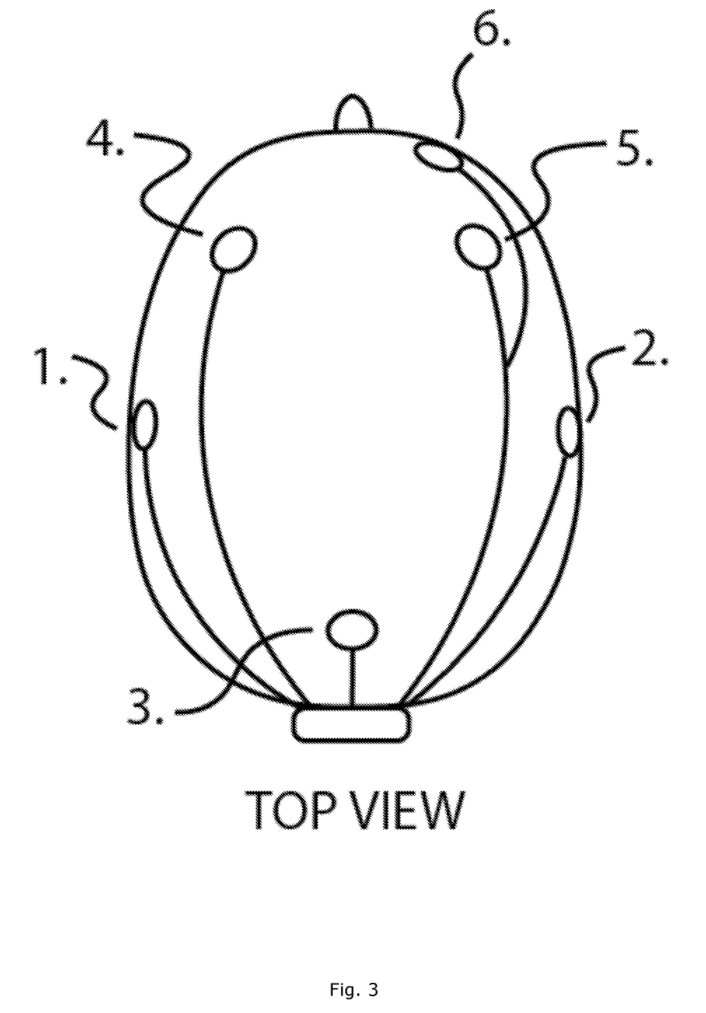

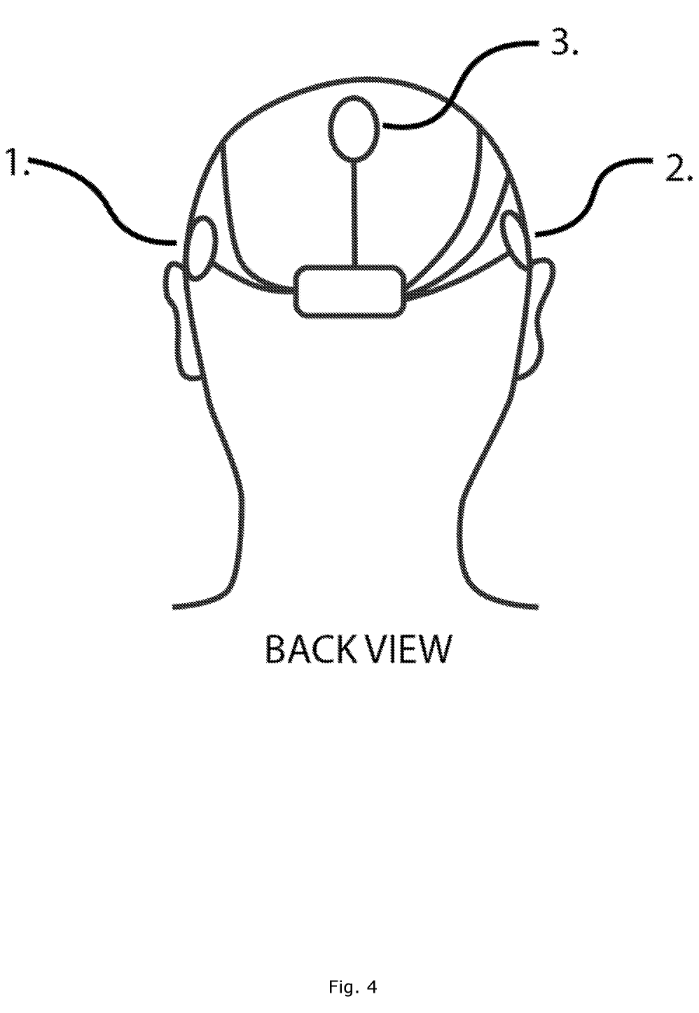

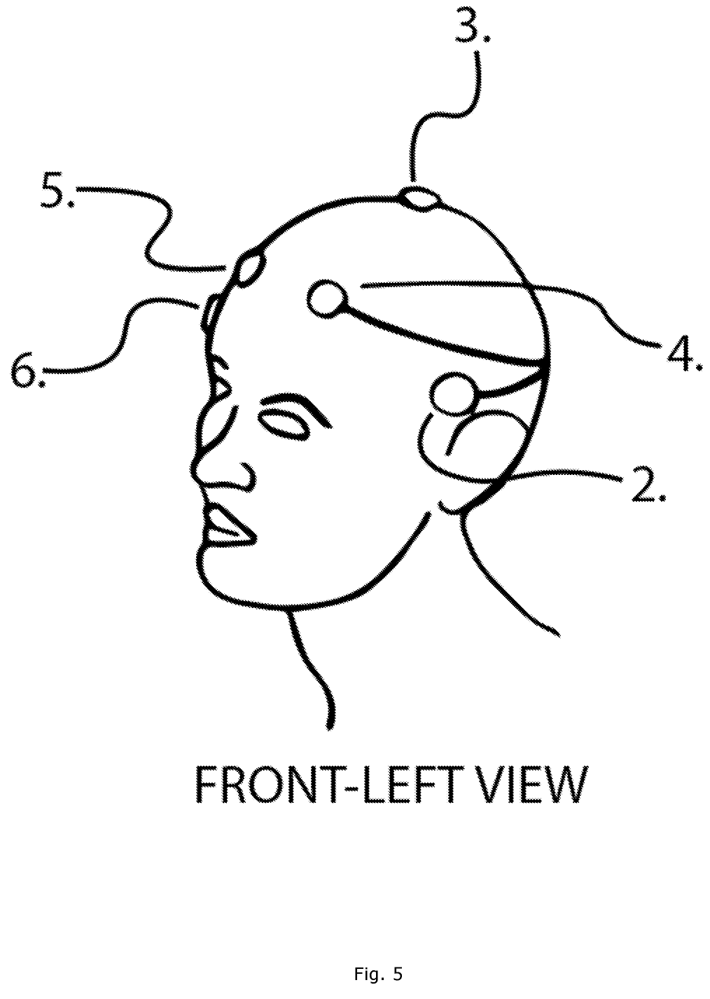

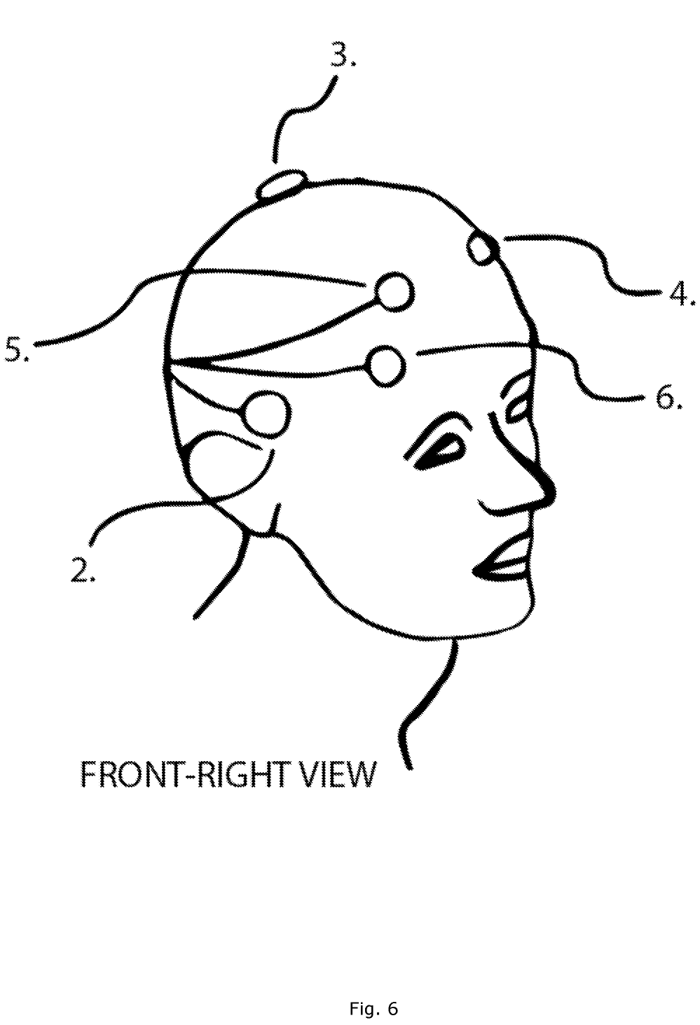

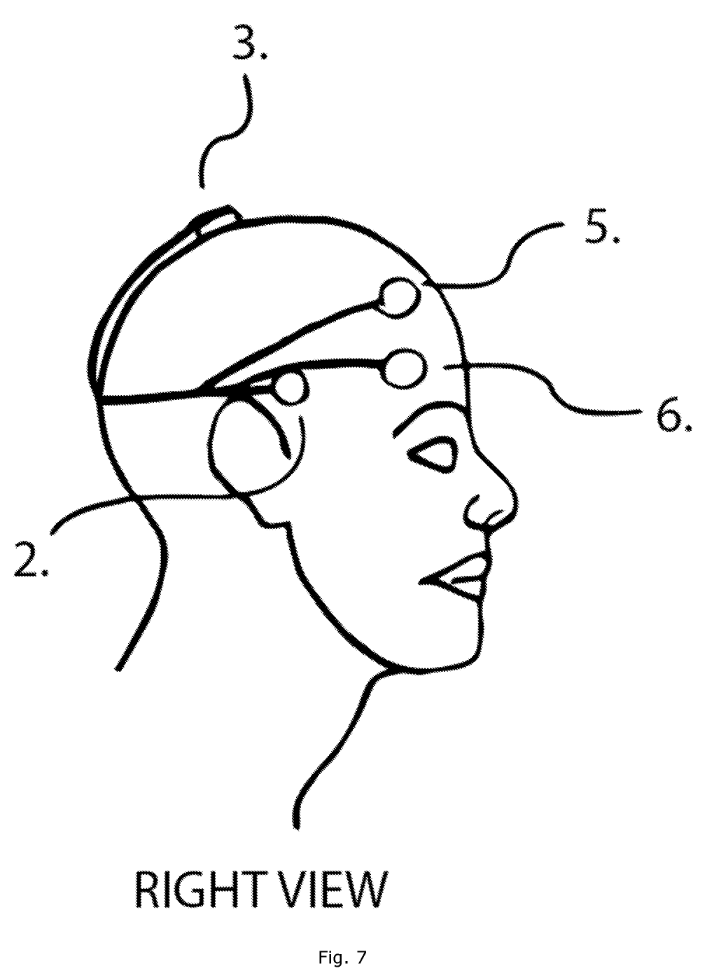

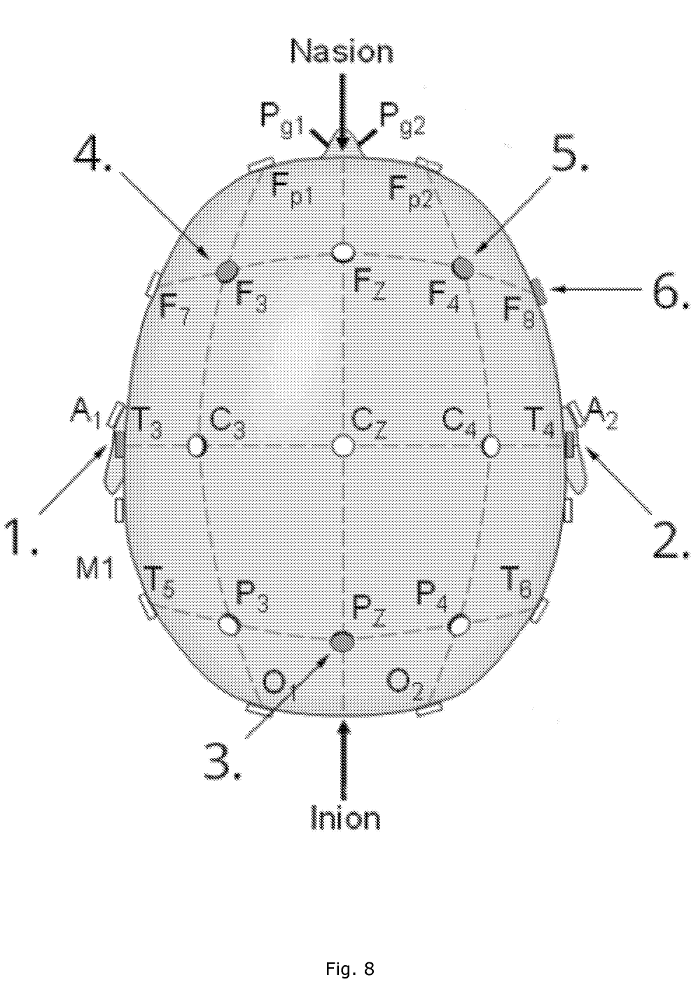

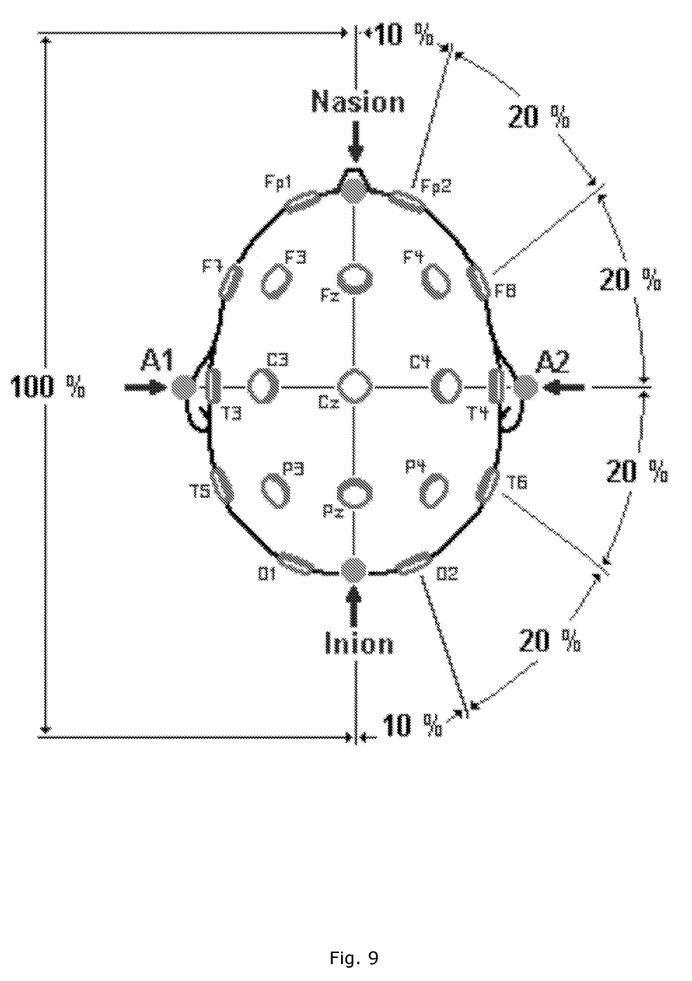

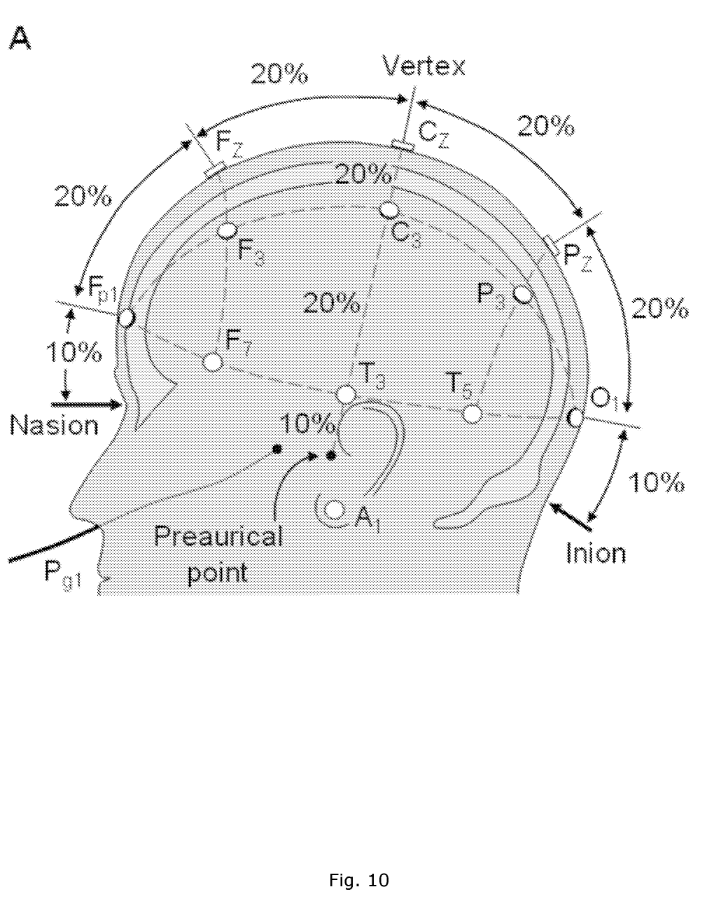

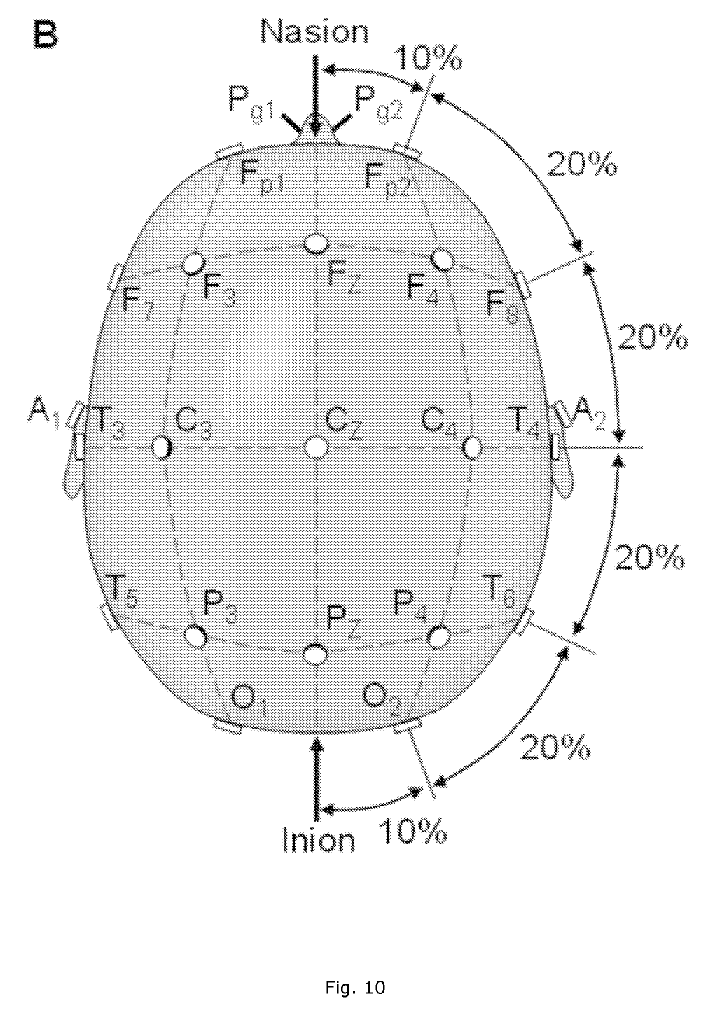

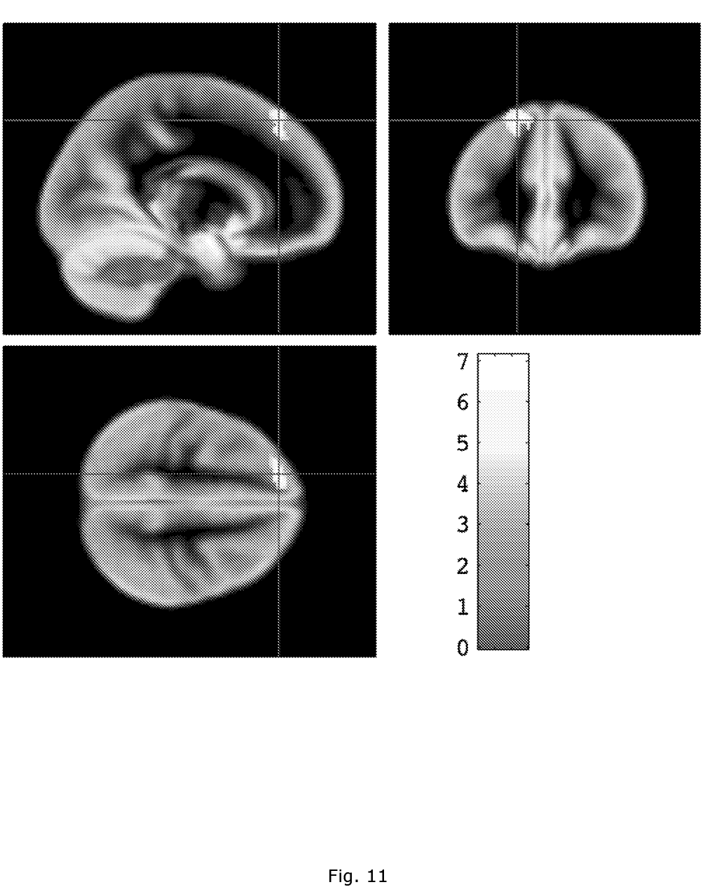

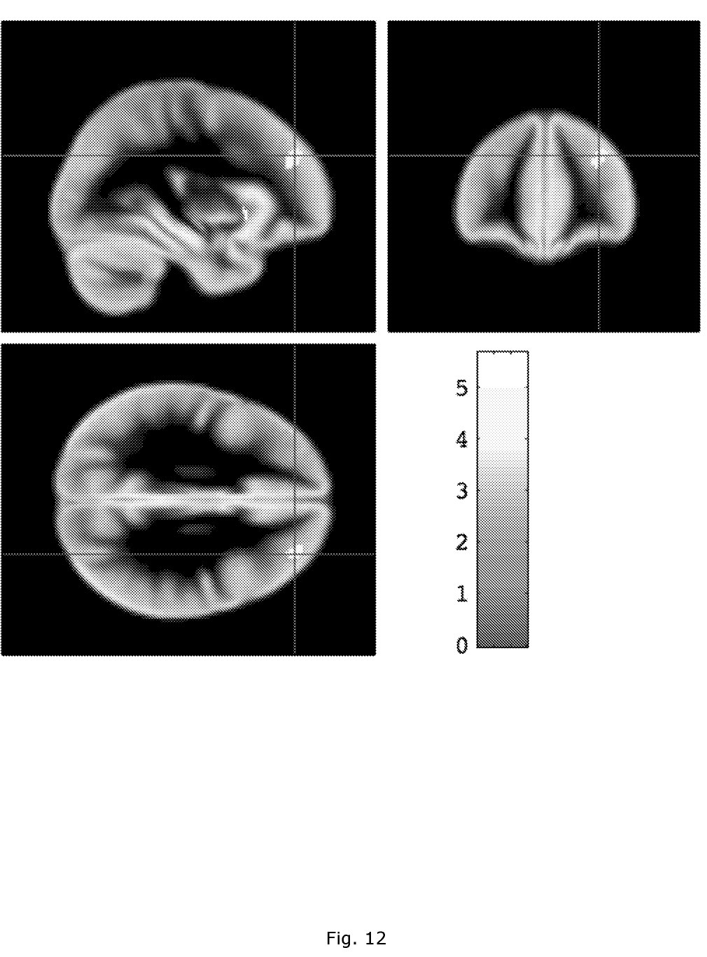

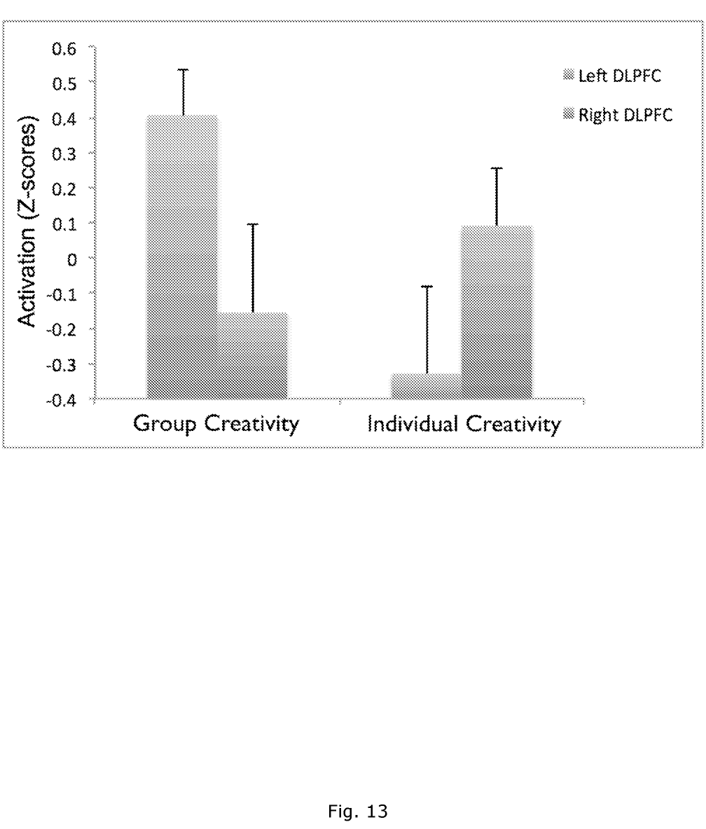

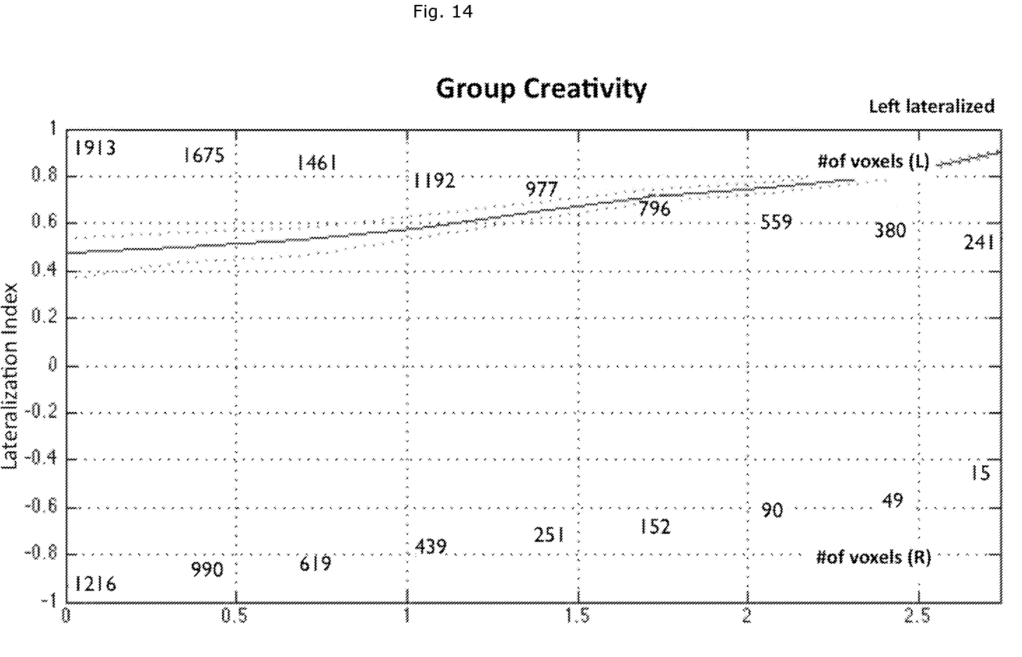

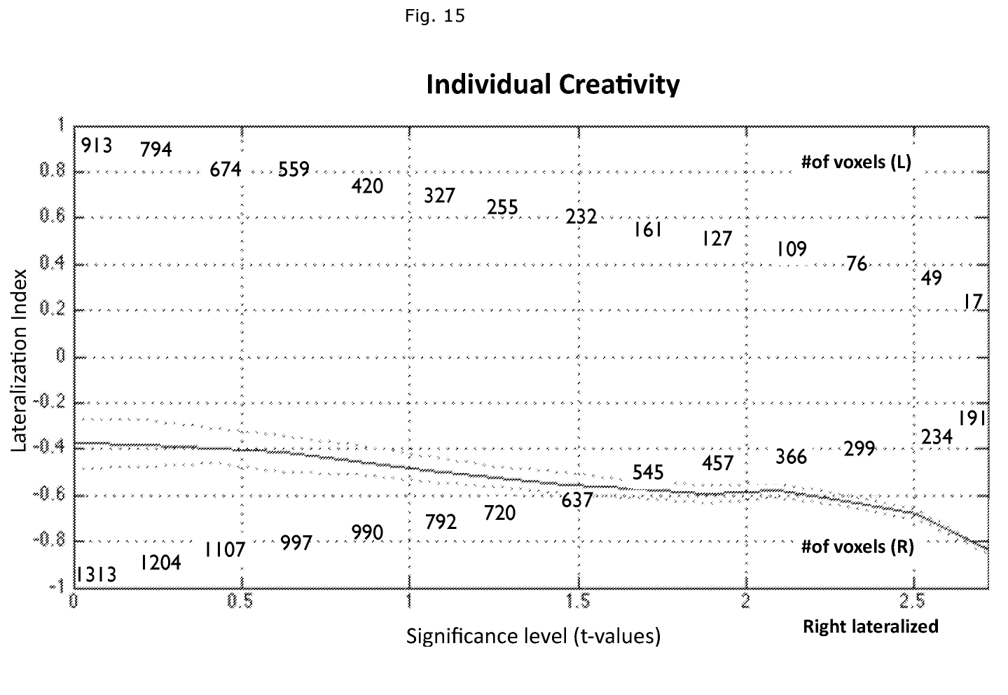

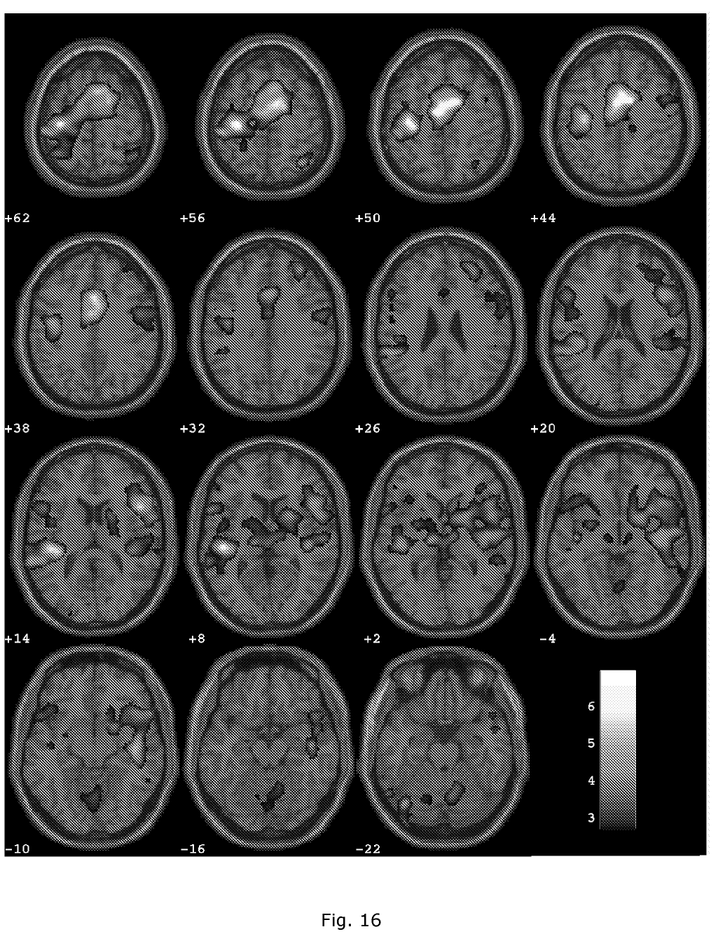

FIG. 2 shows the same device as in FIG. 1, correctly mounted on a human head, seen in top-front view.

FIG. 3 shows the same device as in FIGS. 1 and 2, correctly mounted on a human head, seen in top view.

FIG. 4 shows the same device as in FIGS. 1, 2 and 3, correctly mounted on a human head, seen in back view.

FIG. 5 shows the same device as in FIGS. 1-4, correctly mounted on a human head, seen in front-left view.

FIG. 6 shows the same device as in FIG. 1-5, correctly mounted on a human head, seen in front-right view.

FIG. 7 shows the same device as in FIG. 1-6, correctly mounted on a human head, seen in right view.

FIG. 8 shows the placement of multipurpose electrodes from FIGS. 1-7 in accordance with the international 10-20 EEG coordinate system.
1 is a multipurpose electrode targeting the Left Temporal lobe through a position over T3 in the international 10-20 EEG coordinate system,
2 is a multipurpose electrode targeting the Right Temporal lobe through a position over T4 in the international 10-20 EEG coordinate system,
3 is a multipurpose electrode targeting the Precuneus through a position over Pz in the international 10-20 EEG coordinate system,
4 is a multipurpose electrode targeting the Left Dorsal Lateral Prefrontal Cortex through a position over F3 in the international 10-20 EEG coordinate system
5 is a multipurpose electrode targeting the Right Dorsal Lateral Prefrontal Cortex through a position over F4 in the international 10-20 EEG coordinate system
6 is a multipurpose electrode targeting the Lateral Orbitofrontal cortex through a position over Fp2 in the international 10-20 EEG coordinate system.

FIG. 9

Shows the calculation of the positions in the 10-20 EEG coordinate system, seen from a top view of a human head

FIG. 10

Shows the calculation of the positions from 10-20 EEG coordinate system, seen from the left side of a human head (letter A) and top view (letter B).

FIG. 11

Group creativity. Voxels showing greater fMRI signal (p<0.05) for response improvisation than the control condition (imitate) are overlaid a gray-matter whole-brain template, and displayed in orthogonal projections (top-left: Sagittal slice, top-right; coronal slice, bottom-left; transverse slice). The blue cross goes through the peak significant voxel in the left dorsolateral prefrontal cortex and outlines the three orthogonal MRI volume cuts. The color scale shows t values.

FIG. 12

Individual creativity. Voxels showing greater fMRI signal (p<0.05) for free improvisation than the control condition (main meter) are overlaid a gray-matter whole-brain, and displayed in orthogonal projections (top-left: Sagittal slice, top-right; coronal slice, bottom-left; transverse slice). The blue cross goes through the significant peak voxels in the right dorsolateral prefrontal cortex and outlines the three orthogonal MRI volume cuts. The color scale shows t values.

FIG. 13

Activation levels (normalized beta values) in the left and right dorsolateral prefrontal cortex for the individual creativity and group creativity condition. Error bars show standard error of the mean.

FIG. 14

Lateralization index (LI) curve of activation across the DLPFC in the group creativity condition. The dotted lines indicate LI min and LI max. Y-axis: lateralization index (left−right/left+right). Positive values=left lateralized. Negative values=right lateralized. X-axis: thresholded t-value.

FIG. 15

Lateralization index (LI) curve of activation across the DLPFC in the individual creativity condition. The dotted lines indicate LI min and LI max. Y-axis: lateralization index (left−right/left+right). Positive values=left lateralized. Negative values=right lateralized. X-axis: thresholded t-value.

FIG. 16

Whole brain analysis of regions activated in the [Response Creativity]−[Control] contrast for p<0.05. The images are oriented with the anterior at the top and the left side to the reader's left. The numbers beneath each image represents the millimetres above or below a transverse plane running through the anterior and posterior commissures. The colour scale shows the t-values.

### EXAMPLES

### Example 1: Improvisation

In example 1 we present findings from a brain scanning study performed to investigate the brain activity and brain activity involved in individual free improvisation versus response improvisation, as a measurement for individual and group creativity.

Study Design

The study was performed using functional Magnetic Resonance Imaging (fMRI). Neuroimaging recordings were conducted on a 3-Tesla whole-body MR system with a GE 8 channel HD head coil (GE Signa 3.0 Tesla HDx—Twinspeed gradient system, Milwaukee, United States of America). It involved 27 neurologically healthy male musicians with a mean age of 28 (range 24-36). All participants were professional musicians and had normal hearing and were right-handed, as confirmed by the Edingburgh Handedness Inventory (Oldfield, 1971).

None were taking medication or had any history of neurological or psychiatric illness.

Data from two subjects were discarded due to normalization errors (i.e. abnormal brain size precluded normalizing of imagery data onto the standard brain, MNI template) and one subject did not participate in experiment B due to a power failure.

The remaining 22 subjects were all professional musicians or students at the Royal Academy of Music, Aarhus, Denmark. All participants were rhythmically educated (as opposed to e.g. classically educated) and instrument specializations were as diverse as: four bass players, five drummers, three guitarists, one violinist, four saxophonists and one trumpet player. According to self-reports they practiced on average two hours per day (range 0-5). Of the 22 subjects, 21 had received musical training since early childhood ranging from 1 to 12 years of age with a mean age of 7 and one beginning at the age of 18. All except two considered improvisation as effortless and ‘the most natural thing’ and often experienced euphoria from playing. 21 reported to improvise on a daily basis and one on a weekly basis.

All were professional musicians and thus expected to have a high level of creative expertise.

### Experiment A—Individual Creativity

In the first experiment participants listened to a classical vamp (a vamp is a familiar sequence of cords, which provides the performer with the harmonic framework upon which to improvise) and were asked to either tap the main meter of the vamp or to freely improvise to it. Across experiment one, there were a total of 64 trials per participant of either the [Main Meter] or [free improvise] condition. Each trial lasted for eight seconds and was always followed by a jittered [Rest trial] lasting between three and nine seconds.

### Experiment B—Group Creativity

The second experiment was constructed the same way as the first, asides from improvisations being performed to simulate group creativity. Subjects first heard an improvisation performed by a ‘group’ member and were then asked to either: tap the main meter [Main Meter], imitate the improvisation heard [Imitate] or improvise an answer [Response Improvisation]. With the same approach as in the first experiment, each condition had a duration of eight seconds, but differed in that participants listened the first four seconds (the call) and responded in the following 4 seconds (the response), in accordance with the task condition. As in the first experiment, each trial was followed by a jittered [Rest] trial lasting between three and nine seconds. Throughout each trial a metronome, in corresponding tempo, was presented in both the call and response situation.

Statistical Analysis of the fMRI Data

Across all participants, a total of 808704 brain slices were obtained (39 every third second, 33696 per subject). These raw data were reconstructed and morphed into multislices volumes (39 slices for each volume) while converted into analyze format and spatially realigned and unwarped to correct for head movements during acquisition according to all six motion parameters. Subsequently, images were slice time corrected and normalized to the MNI template using SPM5 (Statistical Parametric Mapping, Welcome Department of Imaging Neuroscience, University College London, London, UK; http://www.fil.ion.ucl.ac.uk) executed in MATLAB (Mathworks Inc., Sherborn, Mass., USA). After normalization, the images were smoothed with a 10-mm full-width at half maximum Gaussian filter. In both experiments a design matrix was made, including separate regressors with onsets for each musical trial and each type of musical task. In the second experiment, calls and responses were defined as separate regressors. All events were modeled using the standard hemodynamic response function of SPM5. The model fit to the data was estimated for each participant, using a general linear model (Friston et al., 1995b) with a 128-s high-pass filter, and AR(1) modeling of serial correlation. Individual one-tailed students t-tests related to the different task effects and response time modulation were performed across all contrasts for each voxel in the brain image and, by a statistical parametric map, transformed into Z statistics.

For both experiments, we report activations that were significant at p<0.05, using a false discovery rate (FDR) analysis (Genovese, Lazar & Nichols, 2002). With this method, results were corrected for multiple comparisons and a threshold was set to control the rate of false positives. The FDR is the fraction of false positives among all tests declared significant. Thus, a threshold of p<0.05 means that, on average, less than 5% of the suprathreshold voxels are not truly active.

To determine the anatomy locations of the activation peaks SPM MNI coordinates were converted into Talairach coordinates, using the Matlab WFU Pickatlas version 2.4 (Maldjian & Laurienti, 2003, 2004), and pinpointed to nearest gray matter using the Talairach Daemon (Lancaster et al., 2000) integrated in the WFU Pickatlas.

Main Findings

When comparing the two studies we found that when subjects were engaged in the group-creative task, only the left Dorsolateral Prefrontal Cortex (LDLPFC) was significantly more activated compared to control. For the individual-creative task we found activation of the same area of the brain but in the opposite hemisphere, the right Dorsolateral Prefrontal Cortex (RDLPFC), as pictured in FIGS. 11 and 12.

### Example 2: Analogue Prototype

To test all the technical descriptions in the above patent, an analogue prototype of the device has been built. The prototype consists of a headset like the one pictured as letter D in FIG. 1, but with the distribution unit (letter A in FIG. 1) detached from the headset and connected to the headset with a jack stick cable. The distribution unit utilized a switch board like the one described in the present application, where the desired current for an electrode, anode, cathode, ground or high impedance is routed via a pair of bipolar junction transistors with a common collector connected to the electrode lead. The base of the transistors are connected and enabled by the logic circuit. Each combination in the 2 bit space maps to a state in the electrode.

This enables the system to dynamically pick the appropriate mode for each electrode during a session. The headset is equipped with 6 multipurpose wet-electrodes, electrodes like the ones described herein, consisting of a sponge, a conductive grid and a shell. The sponge is 10 mm thick (wet state), circular and has 16 cm² in area. On one side, the sponge touches skin or hair on the scalp, and on the other side, it has contact to the conductive grid. The purpose of the grid is to regulate and spread out the electricity going through the grid. The shell that has inlet for wires regulates itself to the angle/shape of the scalp and holds the grid and the sponge together. In order to increase the connectivity/conductivity of the electrode, a saline solution is used to soak the sponge prior to use. This saline solution ensures a conductive contact with the skin or hair, and an equal distribution of current on the surface of the head.

The purpose of the prototype was to test the following key functions described herein:
Wiring of multipurpose electrodes
Instant switch between functionality of electrodes
Programming of sequences
Distribution of electricity in electrode surface
Connection between electrodes and the skin
Resistance testing over electrodes
Electrical buildup

First step in the device testing was testing the wiring of the electrodes. This was done by testing the electrical output per electrode using a digital multimeter. The input in the distribution device was 9 voltages at 1.3 milliampere, and the output per electrode was measured to the same.

The second step of testing was that of the instant switch between functionality of electrodes. This was done for all electrodes, one at the time, using the sequential electrode as ground. The sequence was electrode 1, 2, 3, 4, 5, 6, using the following electrodes as the opposite charge in the same sequence: 2, 3, 4, 5, 6, 1. For each electrode, the following sequence, with instant switches was successfully tested: Anode, cathode, deactivation. The ground function was tested in the following sequence when the electrodes functioned as ground for the other test electrode.

The third step of testing was a pre-programmed sequence of activation of electrodes. This test was programmed using an Arduino Uno board, and tested a program consisting of two stimulation sequences:
[360:1,3:1c:2a:30:40:50:60]
[360:1,3:10:2c:30:4a:50:60]

Requiring a direct switch between functionality for electrode 1 (from anode to deactivation), electrode 2 (from anode to cathode) and electrode 4 (from deactivated to anode). The program was tested using three multimeter, and the current, time, activation and polarity were all measured to follow the program.

As described, also the distribution of electricity in the surface of the electrodes is crucial for the functionality of the product, and this was also tested. For each electrode, a random surface sampling of 30 points using the digital multimeters. For all electrodes, all samples unanimously displayed the same output as input, demonstrating that the distribution of the current was even.

In the next step of the testing, the headset was placed on the head of a test subject to measure the connection between the electrodes and the skin of the subject. The electrodes were soaked in saline as described. Using the distribution unit, it was tested that, when fitting the electrodes close to the head of the subject the resistance over two electrodes was in the range of 10 to 30 kilo Ohm, which is considered a sufficiently low resistance. The testing was repeated for each of the electrode pairs. The test was made without full active stimulation, and can be repeated prior to active stimulation to measure whether the electrodes are correctly placed.

Following the testing of resistance between electrodes when connected to the skin of a human subject, the test was repeated using active stimulation circuits. The purpose of this test was to test the drop of current across the head, measured as a function of the active stimulation, as part of a sequence. This measurement was programmed as part of the active stimulation, and demonstrates how the resistance in the head and following drop of current can be constantly measured in the distribution unit, and if necessary used to adjust the flow of current accordingly.

The final element of the prototype testing was the programming of the increasing intensity of currents. Here, the current was delivered to the electrodes with an increasing intensity, starting at 0 and building up to the full intensity over time. The benefit of this increase in intensity is two-folded: it both makes the application of current less painful for the subject and it makes the stimuli less abrupt for the brain.

In this test, the max intensity was 2 milliampere, and the increase per second was 0.001 mA per second starting at 0.001 at time 0 reaching full intensity after 210 seconds. The test was performed with the headset mounted on a human subject, and the intensity of the stimulation was tested over the electrodes using a digital multimeter. The build-up was clocked on a digital timer, and tested 5 times on different subjects with no errors in increase being detected.

### Example 3: Three Multipurpose Electrode Stimulation

To further test the use of the device, an experimental test has been conducted using the prototype described in Example 2 above. To simplify the testing, only three electrodes were involved in the experiment, utilizing only two general modes of creative problem solving: divergent or convergent thinking.

The study was performed in single sessions, using the prototype to stimulate one human subject at the time. It involved 16 neurologically healthy test subjects, all right-handed, as confirmed by the Edinburgh Handedness Inventory (Oldfield, 1971). None were taking medication or had any history of neurological or psychiatric illness. All subjects were native Danish speakers, and test information and the test setup sought to test whether neurostimulation of the cognitive processes involved in divergent thinking would increase the subject's ability in classical divergent thinking tests. As a control measure convergent thinking tests with a within-subject design were used. According to the research presented in the patent, the aim of the test was to stimulate the right Dorsolateral Prefrontal Cortex positively (V+RDLPFC), while simultaneously stimulate the left Dorsolateral Prefrontal Cortex negatively (V−LDLPFC). As described in the research section of the patent, when the prefrontal area is activated for divergent thinking, Precuneus should also be stimulated positively. In the experiment, this was done by stimulating Precuneus positively (V+Precuneus) utilizing LDLPFC as cathode (V−LDLPFC).

The experiment was performed using transcranial Direct Current Stimulation (tDCS), with 9 volts delivered at 1.3 milliampere to an electrode with a surface area of 16 square centimetres. This gives a surface stimuli equal to 62.5 microampere per square centimetre. Current tDCS research paradigms operate with a surface stimuli of up to 80 microampere per square centimetre, and stimuli duration of up to 30 minutes per test subject per day, with a maximum of 5 stimulations per 7 days.

To achieve the desired stimulation of the above-mentioned areas, three multipurpose electrodes were used. The locations of the three was:

T3 in the 10-20 system, targeting LDLPFC

T4 in the 10-20 system, targeting RDLPFC

Pz in the 10-20 system, targeting Precuneus

The desired combination was to first stimulate V+Precuneus V−LDLPFC for 7 minutes, followed by V+RDLPFC V−LDLPFC for 20 minutes. This gave the following code program containing two sequences:

[420:1,3:T3C:T40:PzA]

[1200:1,3:T3C:T4A:Pz0]

To test for divergent and convergent thinking, four cognitive tests were given each participant before stimulation (pre-tests), and four tests during stimulation (peri-tests). The peri-tests were allocated after the first five minutes of stimulation to ensure that the stimulation was having an effect before peri-testing. The test battery consisted of two standard tests for divergent thinking and two for convergent thinking. Each participant thus received two divergent and two convergent tests before stimulation, and two different versions of the same four tests during stimulation. To account for potential differences between the two versions of the tests given per participant, the versions of each test were randomised and counter-balanced across participants.

The two divergent tests used were Verbal fluency (VF) and Alternative/Alternate uses (AUT), and the two convergent tests used were Working memory (WM) and Remote associates test (RAT).

In addition to these four quantitative tests, all participants were interviewed after the tests and stimuli. Out of the 16 users, 3 reported no effect of the stimulation and one got dizzy. For the remaining, who could feel the stimulation, 75% used concepts such as ‘free flow’, ‘no barriers’, ‘lots of thoughts’, ‘easier to remember’, ‘faster access to memory’ to describe the effects of divergent thinking stimulation. One subject even reported that he suddenly had access to memories he normally could not remember. It must be specified here that the subjects did not know which type of stimuli they received. For the convergent thinking stimulation, 67% gave feedback using concepts such as: ‘more focused’, ‘could sense the correct answer’, ‘it felt right’ etc. When asked about how they performed on the different tasks, participants reported that some of the tests felt easier during stimuli, while others seemed harder. The tasks reported as ‘easier’ were unanimously the divergent tasks while the ones reported as ‘harder’ were all convergent tasks.

For the quantitative tests, subjects performed significantly better on the divergent VF task, when stimulated with divergent thinking compared to convergent thinking stimulation (p=0.046, paired t-test). For the convergent WM task, subjects performed significantly better when stimulated with convergent thinking than divergent thinking (p=0.02, paired t-test). For both the AUT and RAT tests we found a positive trend (each congruent to the stimulation given) that did not reach significance. With a larger number of subjects, these effects would presumably have reached significance.

In conclusion, the study presented above showed that participants performed better on divergent thinking tasks with divergent stimulation and better on convergent tasks with convergent stimulation. For the subjective experience reported by the participants, this was in agreement with the stimulation given. Both the qualitative and quantitative data support that the stimulation worked well, although not all participants felt an effect.

### Example 4: Accumulation of Chlorine at the Anode

When current is applied through a saline solution applied to the sponge on the electrode surface, the charged ions will migrate at the two electrodes involved in a circuit. Chlorine ions will migrate to the anode, where they can form Chlorine gas. Thus, to ensure a non-poisonous environment at the electrode surface, the amount of chlorine gas formed at the anode was calculated:

The theoretical amount of Chlorine gas depends on the current level and the time (Q=I*t). To calculate the theoretical amount of Chlorine gas at the anode, the following assumptions were used:
Chlorine ions are the only ions affected by the current
Current level: 2 mA
Time: 30 min

Results:

Using (Q=I*t) with these assumptions result in approx. 0.001347 g Cl2 which can theoretically be formed at the anode, when current is at 2 mA for 30 min and Chlorine ions are the only ones affected. This amount of Chlorine gas is sufficiently low to ensure a non-poisonous environment at the electrode surface and thus on the subject's skin.

### Example 5: Gas Development at the Electrodes

Since the sponges are soaked in saline, it is theoretically possible that there will develop hydrogen, oxygen and chlorine gas when current is applied.

Experiment aim: To investigate if gasses are formed at the electrodes.

Test: An electrolysis experiment was done, with the preferred saline solution (0.1M NaCl), current level (2 mA) and two 20 cm2 TCT sponges.

Result: According to the results from the electrolysis, no gasses were formed at either the anode or the cathode, based on a visual control of lack of bubbles produced on the electrode surface.

### Example 6: pH for the Saline Solution

As the saline soaked sponges on the electrode surface are in direct contact with the skin, it is important that the pH is within the range of natural pH levels in regular tap water which is considered safe for application on the skin.

Aim: To ensure that the pH of the preferred saline solution (0.1M NaCl) is within the natural pH range of tap water (≈8 pH).

Test: Measuring the saline solution pH with universal pH-paper

Results: The pH of the 0.1M NaCl saline solution was found to be 8, the same pH value as tap water.

### Example 7: The Reason for No Gas Formation—Salt Concentration

From the electrolysis experiment in example 5, it was seen that no gasses where formed at any of the electrodes. Thus, it was desired to investigate whether a higher salt level in the saline solution would develop gas.

Aim: To determine if a higher salt concentration would develop gas.

Test: Electrolysis with a higher salt concentration (>0.1M NaCl), but the same current level (2 mA) and the electrodes used in the device.

Result: No gasses where formed, independent of the salt concentrations tested, thus the lack of gas formations is not dependent on salt level in the saline.

### Example 8: The Reason for No Gas Formation—Current Level

From example 7, it was found that no gasses where formed at any of the electrodes, independent of salt concentration. Thus another experiment was designed to investigate whether a different current level would result in gas developments.

Aim: To determine if the current level (2 mA) was too low to develop gas.

Test: Electrolysis with the saline solution (0.1M NaCl) and a new 9V battery with a capacity of 400-550 mAh.

Result: Significant amount of hydrogen gas where formed at the cathode, reveling that it was the low current level (2 mA) used in examples 5 and 7 that was responsible for no gas formation.

### Example 9: Amount of Saline Solution Needed for the TCT Sponge

In order to obtain good connection, the sponge needs to be well soaked with saline. However, too much saline solution can result in dripping. If the sponges are dripping, the current will run on the wet surface of the skin, and the surface area of the electrical stimulation is thus not controllable.

Aim: To find the right amount of saline solution, when using the TCT sponges.

Test: Three sponges at different state (1. New sponge, 2. Used washed sponge, 3. Used dry sponge) where moisturized until they were fully saturated but not dripping. Both the amount of saline used for moisturizing the three sponges, and the change in weight (measured before and immediately after moisturizing) were registered, and the sum of all measurements was averaged. The mean of the change was 8 mL.

Result: The average amount of saline solution needed for the three sponges was 8 mL.

### Example 10: Heat Development Due to the Power

When applying current to the head of a subject through electrodes, the power generated from the current can be converted into heat. To investigate the amount of heat generated, an experiment was performed.

Aim: To calculate the highest temperature change for the saline solution.

Conditions:
All the power is converted into heating the saline solution
The saline solution has the same specific heat capacity as water 4187 J/kg*K
Amount of solution 10 mL=0.01 kg
9V and a current at 2 mA are used, given an effect at 0.018 Watt
Time is set for one session=30 min
Start temperature is set at 25° C.

Results: With the conditions listed above the temperature rises only 0.77° C. during a 30 minute session. Thus, heating of the saline solution is not an issue.

### Example 11: Current Density

A high current density can have unpleasant and even harmful effect on the skin of the subject during stimulation. Reported brain lesions occur at e.g. current density 142.9 A/m² based on scaled experiments in rats.

Aim: To calculate the current density at the highest possible current level 2 mA and the smallest expected electrode area of 17.1 cm².

Calculations:

CD[mAcm2]=IA=2 mA/17.1 cm2=0.117 mAcm²

Results: For the highest current level and the smallest surface (the electrode alone), current density was found to be 1.176 A/m², thus giving no reason for concerns in the current invention.

### Example 12: Break Test on TFC Sponges

In longitudinal use of electrodes there is a general concern that the sponge surface will brittle or break, leading to uncontrollable distribution of the current and thus potential ineffective stimulation. Various types of sponges previously tested have been seen to get brittle over time, thus it was desired to test the durability of the preferred TFC sponges.

Aim: Investigate whether the TFC sponges gets brittle.

Test: Soaking a TFC sponge in saline water, thus folding it and setting it to dry. This was repeated 20 times.

Results:

The TFC sponge showed no indication of brittle behaviour.

### Example 13: Resistance Through Different Sponge Types

The ideal sponge for tDCS has as low resistance as possible, thus when choosing a new sponge this is an important parameter.

Aim: To find a sponge with as low resistance as possible.

Test: Measuring the resistance through five difference sponges. The resistance was measured with an Ohmmeter. 150 measurements were done on each sponge, to get a representative average.

Results: The highest resistance was found for a TCT sponge with an average resistance at 605 k Ohms. The TFC sponge had the lowest resistance with an average at 505k Ohms.

### Example 14: TGA Analyzes of the TCT Sponge

In one embodiment the invention uses TCT sponges on the electrode surface. The sponge seems moistened when unpacked, and dries out after being soaked in saline solution.

Aim: To determine the amount of water in a new and a used sponge.

Test: Thermal gravity analysis was done on a new and a used sponge. TGA increase the temperature with 20° C. pr. min. from 0° C. up to 900° C.

Results: More than 25% of the original weight is lost before the temperature reaches 100° C., which indicates that 25% of the original weight is water in a new sponge. A used unwashed sponge contains approximately 10% water.

### Example 15: Discomfort Level

During tDCS it is common to experience itching and mild discomfort.

Aim: To determine whether it is the current or the saline solution that is responsible for the discomfort.

Test: Volunteer who experienced significant discomfort in a previous test of electrical neurostimulation. Wore a neurostimulation device with saline soaked sponges, without any current flowing.

Results: No discomfort associated with using the invention without applying any current. Thus, the reason for the discomfort is not the saline solution, but the applied current.

### Example 16: Connection Only with Tap Water

Traditionally saline solution is used in tDCS to ensure good connection. However, tap water also contains ions, which can work as current carriers.

Aim: To determine if tap water is conductive enough for tDCS.

Test: Soaking the sponges in tap water and monitoring the connection on the app.

Results: The connection was significant lower and unstable, when using only tap water. Thus, a saline solution is preferred for the wet electrode use.

### Example 17: Size Change of the Sponge

When dried, the TFC sponge shrinks up to 21% of the original size.

Aim: To ensure that the size change does not became permanent.

Test: The TFC sponge was soaked in saline, measured and set to dry, then measured again in dry state. This procedure was repeated 20 times.

Results: There were no indications that the number of repetitions had any effect on the size changes. Furthermore the TFC sponge resumed its original size ever time it was soaked, showing to result in permanent changes.

### Example 18: Theoretical Amount of Salt Affected by the Current

The theoretical amount of salt that is affected by the current, can be calculated based on the value of amps and time (Q=I*t).

Aim:

Calculating the amount of salt affected by the current

Assumptions:

It is only the salt ions that are affected by the current

2 mA

30 min

Results:

Approximately 0.001347 g Cl₂ will theoretically migrate to the anode. Based on this, the applied current only affects approximately 0.00216 g of NaCl, rounded up to 0.022 gram in 100 ml of water, which is the minimum salt concentration needed for the prescribed stimulation.

1. A system for applying a weak electrical current to a human brain or nervous tissue, wherein the system comprises:
a) at least three multipurpose electrodes applied in a fixed position on the head of a human subject, wherein each electrode is wired to interchangeably function as anodal stimulation, cathodal stimulation, deactivation, or ground, and
b) a distribution unit configured to provide the anodal stimulation, cathodal stimulation, deactivation, or ground,
wherein at least two of the multipurpose electrodes are disposed on the right hemisphere and at least one is disposed on the left hemisphere.

2-23. (canceled)

24. The system according to claim 1 further comprising a control unit having an interface configured to communicate with the distribution unit.

25. The system according to claim 1, wherein the control unit is configured to provide a predetermined signal to the distribution unit.

26. The system according to claim 25, wherein the predetermined signal comprises predetermined sequences.

27. The system according to claim 1, wherein the distribution unit is configured to provide a weak electrical current of less than 3 mA per electrode.

28. The system according to claim 27, wherein the weak electrical current provided per electrode has the same intensity.

29. The system according to claim 27, wherein the weak electrical current provided per electrode has a different intensity.

30. The system according to claim 27, wherein the distribution unit provides the weak electrical current per electrode as anodal stimulation, cathodal stimulation, deactivation, or ground.

31. The system according to claim 1, wherein one electrode is placed over the left ear at position T3 on the 10-20 EEG placement scheme.

32. The system according to claim 1, wherein one electrode is placed over the right ear at position T4 on the 10-20 EEG placement scheme.

33. The system according to claim 1, wherein one electrode is placed at the top of the head at position Pz on the 10-20 EEG placement scheme.

34. The system according to claim 1, wherein one electrode is placed at the left side of the forehead at position F3 on the 10-20 EEG placement scheme.

35. The system according to claim 1, wherein one electrode is placed at the right side of the forehead at position F4 on the 10-20 EEG placement scheme.

36. The system according to claim 1, wherein one electrode is placed over the right eye at position Fp2 on the 10-20 EEG placement scheme.

37. The system according to claim 1, wherein the distribution unit is configured to receive electrical signals from each electrode.

38. The system according to claim 1, wherein the control unit is configured to detect electrical signals and digitalize recorded signals.

39. The system according to claim 1, wherein the system is configured to analyze recorded digital data.

40. The system according to claim 1, wherein a configuration or settings obtained from recorded digital data is returned to the control unit.

41. The system according to claim 1, wherein at least one fuse has an individual ampere fuse or a voltage fuse is placed between an electrode and the distribution unit.

42. The system according to claim 1, further comprising at least two fuses, which are individual ampere fuses or voltage fuses, wherein one fuse is placed at the wire or electrode connection and one fuse is placed either at the wire or distribution unit connection or is incorporated into the distribution unit.

43. The system according to claim 1, wherein at least one of the electrodes is a dry electrode.

44. A method for inducing a cognitive effect in a human subject comprising:
contacting a human subject with the system of claim 1 and inducing a cognitive effect in said human subject.

45. A method of inducing a predetermined cognitive effect in a human subject, the method comprising:
a) applying at least two electrodes on the right hemisphere and at least one electrode on the left hemisphere, wherein the electrodes are wired to interchangeably function as anodal stimulation, cathodal stimulation, deactivation, or ground; and
b) applying a weak electrical current having predetermined flows or patterns to the brain of the human subject, thereby achieving the predetermined cognitive effect in said human subject.
